(19) United States
(12) Patent Application Publication
(10) Pub. No.: US 2021/0214782 A1
(43) Pub. Date: Jul. 15, 2021

(54) METHOD OF USING GENETIC MARKERS, SINGLE NUCLEOTIDE POLYMORPHISMS AND/OR INDELS TO DETERMINE RESPONSIVENESS TO IL-10 OR IL-10 DERIVATIVE TREATMENT

(71) Applicant: Deka Biosciences, Inc., Bethesda, MD (US)

(72) Inventor: John MUMM, Bethesda, MD (US)

(21) Appl. No.: 17/145,169

(22) Filed: Jan. 8, 2021

Related U.S. Application Data
(60) Provisional application No. 62/959,025, filed on Jan. 9, 2020.

(51) Int. Cl. C12Q 1/6869; C07K 14/54; C12Q 1/6827

(52) U.S. Cl. CPC C12Q 1/6869; C07K 14/5428; A61K 38/00; C07K 2319/30; C12Q 1/6827

(57) ABSTRACT

The application relates to the discovery of novel gene expression profiles and/or single nucleotide polymorphisms (SNP) and/or insertions or deletions of bases (INDEL) profiles that correlate with a subject's positive receptiveness to Interleukin 10 (IL-10) or IL-10 based agent treatments. The application also relates to methods of treating patients with an IL-10 or IL-10 based agent treatment by screening, examining, or determine patients possessing a gene expression profile and/or SNP and/or INDEL profile most receptive to the treatment.

The various embodiments described in this application relate to the discovery of novel gene expression profiles and single nucleotide polymorphisms (SNP) and/or insertions or deletions of bases (INDEL) profiles that correlate with a subject's positive receptiveness to Interleukin 10 (IL-10) or IL-10 based agent treatments. Other aspects described in this application include methods of identifying the gene profiles or expression profiles and, SNP and/or INDEL profile(s) indicative of a subject's responsiveness to IL-10 or an IL-10 based agent, as well as methods of screening for subjects that are more receptive to IL-10 or IL-10 based agent treatment or those subjects that are less responsive or receptive to IL-10 or IL-10 based agent treatment. This application also describes methods of screening a subject suffering from a cancer indication or suffering from inflammatory disease or disorder and determining, based on certain gene profiles or expression profiles, and SNP and/or INDEL profiles, whether the subject will be receptive to IL-10 or IL-10 based agent treatment. Finally, this application describes methods of treating patients with cancer or patients with inflammatory disease comprising first examining, determining, or screening a subject for responsiveness or receptiveness to treatment with an IL-10 or IL-10 based agent and if determined to be responsive or receptive to treatment administering to the patient an IL-10 or IL-10 based agent.

### BACKGROUND

IL-10 is a non-covalent homo-dimeric cytokine with structural similarities to Interferon γ (IFNγ). The IL-10 receptor consists of two molecules of the IL10 receptor 1 (IL10R1) and two molecules of the IL-10 receptor 2 (IL10R2). Moore, 2001. The IL-10 receptor is expressed on the surface of most hematopoietic cells and most highly expressed on monocytes/macrophages and T-cells.

While IL-10 has been reported to be both an immunosuppressive (Schreiber, 2000) and immunostimulatory cytokine (Mumm, 2011), clinical evaluation of IL-10 treatment of Crohn's patients resulted in an inverse dose response (Fedorak, 2000; Schreiber, 2000), whereas treatment of cancer patients with PEGylated IL-10 resulted in dose titratable potent anti-tumor responses. Naing, 2018.

PEGylated IL-10 anti-tumor response requires endogenous CD8+ T cells and IFNγ. Mumm, 2011. Treatment of tumor bearing animals with PEGylated IL-10 results in increased intra-tumor CD8+ T cells and increased IFNγ on a per cell basis.

Due to the pleiotropic effects of IL-10, variants of the EBV IL-10 homologue were designed that are significantly reduced in their ability to stimulate IFNγ secretion from activated CD8+ T cells while still retaining their suppressive effects on monocyte/macrophage secretion of pro-inflammatory cytokines. This was accomplished by evaluating 3 differential affinity variants of the EBV IL-10 homologue, termed DV05, DV06, and DV07. U.S. Pat. No. 10,858,412. DV06 exhibits the least CD8+ T cell stimulatory effects alone when fused with the scaffolding diabody technology. Id.

Clinical investigators originally discovered that treatment of Crohn's patients with IL-10 resulted in approximately 24% remission of Crohn's symptoms and inflammation at a dose of 5 micrograms/kg daily dosing. Fedorak, 2000 at FIG. 1a. Higher doses of 10 and 20 micrograms/kg daily resulted in a reduction (from 24%) of patients experiencing remission. All doses (1, 5, 10, and 20 micrograms/kg daily) resulted in the suppression of TNFα secretion from peripheral patient blood treated in vitro with lipopolysaccharide (LPS). Tilg, 2002 at FIG. 1b. Further investigation revealed that peripheral blood from patients treated with 10 and 20 micrograms/kg of IL-10 (daily) secreted higher neopterin (data not shown) and IFNγ when stimulated with phytohemagglutinin (PHA). Id. at FIG. 1c. Similar to the reports that illustrate IL-10's capacity to induce adaptive anti-tumor immunity through induction of IFNγ via activation of CD8+ T cells (Mumm, 2011; Naing, 2018), these data collectively suggest that at higher concentrations, (10 and 20 micrograms/kg) IL-10's predominate immunological effect is stimulatory, rather than suppressive. The data suggests that a differential response to IL-10 of the monocytes/macrophages and CD8+ T cells underlies these different diseases. IL-10's anti-inflammatory response has been reported to depend on regulating monocytes/macrophages (Malefyt, 1991), whereas the anti-tumor response induced by IL-10 has been shown to require both CD8+ T cells and IFNγ, (Mumm, 2011).

Using previously described in vitro monocyte/macrophage (similar to one used by, for example, Malefyt, 1991) and T-cell response assays (Chan, 2015) normal donor monocyte/macrophage and T cell responses to IL-10 were assessed. In monocytes/macrophages, IL-10 suppresses the secretion of pro-inflammatory cytokines (IL-1β, TNFα, and IL-6) in response to stimulation with LPS. The data indicates that concentrations as low as 0.1 ng/mL significantly inhibit monocyte/macrophage responses to pro-inflammatory stimuli. FIG. 1. Administration of IL-10 to donor peripheral blood CD8+ T cells induced IFNγ. FIG. 2. While 0.1-1 ng/mL IL-10 suppresses macrophage/monocyte responses, CD8+ T cells are stimulated to secrete more IFNγ at concentrations higher than 10 ng/mL. The inventor sought to determine whether patients can be classified/stratified based on their response to IL-10, the monocyte/macrophage and T-cell response assays were used to assess both suppressive and stimulatory responses to IL-10 and to determine whether all human monocytes/macrophages and T cells respond to IL-10 in the same manner. The inventor has surprisingly found that there are differential responses in monocytes/macrophages and CD8+ T-cells to IL-10. Further, the inventor has found that the magnitude of response to IL-10 is based on a combination of donor or patient genetic factors that result in differential expression profiles.

### SUMMARY OF PREFERRED EMBODIMENTS

This application describes gene expression profiles and one or more single nucleotide polymorphisms (SNP) and/or insertion or deletion of bases (INDEL) profiles that identifies a subject whose CD8+ T cells are more receptive to an IL-10 or IL-10 based agent treatment. Also described in this application are methods of identifying subjects or individuals, such as those suffering from inflammatory disease, who are more likely to respond to IL-10 or IL-10 based agent reconstitutional treatment. In other aspects, the application relates to methods of identifying gene expression profiles and/or SNP and/or INDEL profiles in subjects who have monocytes/macrophages that phenotypically exhibit low induction or secretion of IL-10 (in response to a pro-inflammatory stimulus) and high response to IL-10 (by way of reducing TNF-α induction or secretion). In other aspects, the gene expression profiles and/or SNP and/or INDEL profiles correlate with a phenotype where a subject's monocytes/macrophages have low production or secretion of IL-10 (in response to a pro-inflammatory stimulus) and increased reduction of TNF-α secretion. Finally, this application describes methods of treating diseases or disorders with IL-10 or an IL-10 based agent by screening for certain gene expression profiles and/or SNP and/or INDEL profiles in subjects who are identified as positive IL-10 or IL-10 agent responders and administering a therapeutically effective amount of an IL-10 or an IL-10 based agent.

Accordingly, an embodiment of the present application includes a method of identifying a gene expression profiles and/or genotyping SNP and/or INDEL profiles to obtain a profile indicative of responsiveness to treatment with an IL-10 or an IL-10 based agent and/or combination of IL-10 with EPO, TGF-beta, basic FGF, FGF, PDGF, IL-4, IL-11, or IL-13, the method comprising exposing a cellular sample from a patient to LPS or a derivative thereof or other Toll-like receptor agonists (e.g., CpG) to elicit IL-10 induction or secretion; and/or measuring the levels of TNF-α production and the levels of IL-10 induction or secretion from the cellular sample; selecting the patient with the sample exhibiting a dual phenotype of (1) high TNF-α reduction (or decreased production of TNF-α) in response to IL-10, IL-10 derivative, or an IL-10 based agent stimulation and (2) low IL-10 induction or secretion, in response to LPS stimulation; and sequencing the entire genome from the patient exhibiting the dual phenotype for the presence of a gene expression profile and/or a SNP and/or INDEL profile.

An additional embodiment of the present application includes a method of identifying a gene expression profile and/or genotyping a SNP and/or INDEL profile to obtain a profile indicative of T cell responsiveness to treatment with IL-10, IL-10 derivatives or an IL-10 based agent or a combination of IL-10, IL-10 derivatives or an IL-10 based agent with IL-2, IL-7, IL-12, IL-15, IL-26, IL-27, IL-28, IL-29, IFN-α based agents, the method comprising exposing a cellular sample from a patient to the above agents and a soluble anti-CD3 at a range of concentrations for a period of time sufficient to illicit the induction of secreted IFN-α. Those patient samples associated with the phenotype of high IFN-α induction from CD8+ T cells in response to IL-10, IL-10 derivatives, or an IL-10 based agent will undergo genetic profiling to obtain gene expression profile and/or SNP and/or INDEL profile. The gene expression profile and SNP and/or INDEL profile associated with high IFN-γ induction from CD8+ T cells in response to IL-10 will be utilized to select patients for treatment with and IL-10, IL-10 derivatives, or IL-10 based agent and/or combination therapy.

Another embodiment of the present application includes determining whether a subject is likely or less likely to respond to treatment with IL-10, IL-10 derivative thereof, or IL-10 based agent comprising sequencing nucleotides from a patient for a gene expression profile, and SNP and/or INDEL profile that results in an indication of reduced TNF-α production and low IL-10 induction or secretion, wherein the reduced TNF-α production is in response to an IL-10 based stimulus and low IL-10 induction or secretion in response to a proinflammatory stimulus.

Other embodiments of the present application include screening a subject for the presence of a gene expression profile and SNP and/or INDEL profile to determine whether the subject is a candidate receptive to receiving IL-10, IL-10 derivative, or IL-10 based agent treatment. Such treatment is for the intended purpose of treating an inflammatory disease, such as but not limited inflammatory bowel disease, Crohn's disease, or ulcerative colitis. Other treatments include any disease or disorder wherein the reduction of monocytes/macrophage inhibition or stimulation is needed.

In another embodiment, the application relates to a method of genotyping a SNP or INDEL to obtain a profile indicative of patient receptiveness to treatment with IL-10, IL-10 derivative, or an IL-10 based agent comprising contacting activated CD8+ T cells obtained from the patient with an amount of IL-10 or an IL-10 based agent to induce secretion of IFN-γ; measuring the level of IFN-γ secretion; selecting the patient sample exhibiting a phenotype of high and/or medium IFN-γ secretion by CD8+ T cells; and sequencing the entire genome from the patient exhibiting the phenotype for the presence of a SNP and/or INDEL profile.

An embodiment of the present application relates to specific genetic markers that correlate with patient, preferably inflammatory disease patients, responsiveness to IL-10. In other embodiments, the present application relates to specific expression profile, and/or SNP and/or INDEL profile(s) that may be useful in identifying select patients who will be more responsive to receiving IL-10 or IL-10 based agent treatments.

In yet another embodiment, the present application relates to a method for treating a patient suffering from an disease or disorder alleviated by IL-10 or an IL-10 agent comprising genetically profiling a sample obtained from a diseased patient for a SNP/INDEL profile associated with responsiveness to IL-10 or IL-10 based treatment. In another embodiment, the method of treating includes selecting an expression profile and/or SNP and/or INDEL profile associated with high TNF-α reduction in response to stimulation form IL-10, an IL-10 derivative, or an IL-10 agent, and low IL-10 induction in response to LPS stimulation; selecting a patient possessing the expression profile, and/or SNP and/or INDEL profile; and administering to the patient a therapeutically effective amount of an IL-10 or derivative thereof, an IL-10 diabody, or an IL-10 mimetic.

In yet another embodiment, the present application relates to a method of treating an inflammatory disease comprising administering to a patient in need thereof a therapeutically effective amount of an IL-10, an IL-10 derivative, an IL-10 diabody, an IL-10 minibody, or an IL-10 agent, wherein the patient possess an expression profile, and/or SNP and/or INDEL profile associated with low IL-10 production in response to a proinflammatory stimulus and high responsiveness to IL-10. In a preferred embodiment, the inflammatory disease or disorder is inflammatory bowel disease (IBD), Crohn's disease, and/or ulcerative colitis.

In yet another embodiment, the present application relates to a method of treating cancer comprising administering to a patient in need thereof a therapeutically effective amount of an IL-10 or an IL-10 based agent, wherein the patient possesses a SNP/INDEL profile based on a gene and protein expression profile provided in Table 1.

In still yet another embodiment, the present application relates to a method of determining whether a subject is likely or less likely to respond to treatment with IL-10 or an IL-10 based agent comprising sequencing a patient cellular sample for a SNP or INDEL profile indicative of high and/or intermediate and/or low IFN-γ secretion by CD8+ T cells. In a preferred aspect, a profile associated with high and/or intermediate IFN-γ secretion by CD8+ T cells (i.e., high and/or intermediate CD8+ T cell response) is indicative of successful treatment of cancer. In another aspect, a profile associated with low and/or intermediate IFN-γ secretion by CD8+ T cells (i.e., low and/or intermediate CD8+ T cell response) is indicative of successful treatment of autoimmune diseases or disorders.

### BRIEF DESCRIPTION OF DRAWINGS

FIG. 1 represents the results of exposing varying doses (0.1, 1, 10, and 100 ng/ml) of IL-10 on monocytes/macrophages and measuring the secretion levels of pro-inflammatory cytokines.

FIG. 2 represents the results of exposing varying doses (0.1, 1, 10, and 100 ng/ml) of IL-10 on CD8+ T cells and measuring the secretion levels of IFN-γ.

FIGS. 3A-3C are schematic representations of various IL-10 variants, such as variants made in EBV IL-10. FIG. 3A(DV05) is an IL-10 variant comprising a single point mutation at amino acid position 31 (V31L). FIG. 3B (DV06) is an IL-10 variant comprising a single point mutation at amino acid position 75 (A75I). FIG. 3C (DV07) is an IL-10 variant comprising a two point mutations at amino acid positions 31 and 75 (V31L and A75I).

FIG. 3D assays monocyte/macrophage response to various forms of IL-10, including wild type human IL-10, EBV-IL-10, DV05, DV06, and DV07. All forms, including the IL-10 variants-DV05, DV06, DV07—suppress TNFα secretion in response to LPS.

FIG. 3E assays T-cell response to various forms of IL-10, including wild type human IL-10, EBV IL-10, DV05, DV06, and DV07. All forms of IL-10 induced IFN-γ secretion from T-cells.

FIGS. 4A-4F are a schematic representations of various configurations of IL-10 fusion protein/immunoconjugate/diabody constructs. FIGS. 4A-4C represent a fusion protein complex (i.e., a diabody) where each fusion protein comprises a VH and VL regions obtained from two different antibodies or a VH/VL pair derived from a single antibody where the CDR regions are grafted from a different antibody to enable the diabody to bind to a different protein from that of the original CDR regions associated with the VH/VL, linked to a monomer of IL-10 (which may also be substituted with an IL-10 variant molecule) via a carboxy terminal linker or an amino terminal linker with FIG. 4A a single mutation—e.g., amino acid position 31—impacting IL-10 receptor binding; FIG. 4B a single mutation—e.g. amino acid position 75—impacting IL-10 receptor binding; and FIG. 4C two mutations—e.g. amino acid positions 31 and 75—impacting IL-10 receptor binding FIGS. 4D-F represent a fusion protein complex (i.e., a minibody) where each fusion protein comprises a single VH or VL region obtained from one antibody linked to a monomer of IL-10 (which may also be substituted with an IL-10 variant molecule) via a carboxy terminal linker or an amino terminal linker with FIG. 4D a single mutation—e.g., amino acid position 31—impacting IL-10 receptor binding; FIG. 4E a single mutation—e.g., amino acid position 75—impacting IL-10 receptor binding; and Figure F two mutations—e.g., amino acid positions 31 and 75—impacting IL-10 receptor binding.

FIGS. 5A-F are a schematic representation of various configurations of the IL-10 fusion protein/immunoconjugate/diabody constructs. FIGS. 5A-C represents a single fusion protein (i.e., minibody) where the monomers of IL-10 (which may also be substituted with an IL-10 variant molecule) are each linked via a carboxy terminal linker or an amino terminal linker to either a VH or VL from the same antibody and the VH and VL are linked together and where the CDR's from a separate antibody have been grafted into the CDR regions to alter the binding capacity from the original CDR's to a protein that is expressed in the tissue of interest. The monomers of IL-10 comprise FIG. 5A a single mutation—e.g., amino acid position 31—impacting IL-10 receptor binding; FIG. 5B a single mutation—e.g., amino acid position 75—impacting IL-10 receptor binding; and FIG. 5C two mutations—e.g., amino acid positions 31 and 75—impacting IL-10 receptor binding. FIGS. 5D-F represents a single fusion protein where monomers of IL-10 (which may also be substituted with an IL-10 variant molecule) are linked together and each monomer of IL-10 is further linked via a carboxy terminal linker or an amino terminal linker to a single VH or VL region obtained from one antibody. The IL-10 monomers comprise Figure D a single mutation—e.g., amino acid position 31) impacting IL-10 receptor binding; FIG. 5E a single mutation—e.g., amino acid position 75—impacting IL-10 receptor binding; and FIG. 5F two mutations—e.g., amino acid positions 31 and 75) impacting IL-10 receptor binding.

FIG. 6A represents the results from an in vitro assay measuring the levels of TNF-α production in monocytes/macrophages in response to stimulation by an IL-10 based agent (DhDe:DV05, DhDe:DV06, DhDe:DV07) as compared to IL-10 and viral EBV-IL-10.

FIG. 6B represents the results from an in vitro assay measuring the levels of IFN-γ production in T-cells in response to stimulation by an IL-10 based agent (DhDe:DV05, DhDe:DV06, DhDe:DV07) as compared to IL-10 and viral EBV-IL-10.

FIG. 7A represents the results from an in vitro assay measuring the levels of IL-10 induction by monocytes/macrophages in response to LPS from various donors. FIG. 7B is an in vitro assay measuring the reduction of TNF-α by isolated monocytes/macrophages in response to IL-10. FIG. 7C is suppression of TNF-α in low producers of IL-10. FIG. 7D is suppression of TNF-α in medium producers of IL-10.

FIG. 8 is a schematic representation of the screening process for determining the gene expression profile and/or SNP and/or INDEL profile in monocytes/macrophages for inflammatory disease.

FIG. 9 represents the results of an in vitro assay measuring the levels of IL-10 induction by monocytes/macrophages in response to LPS in Crohn's patients and the reduction of TNF-α secretion from isolated peripheral blood monocytes/macrophages in response to IL-10 in Crohn's patients.

FIGS. 10A and 10B represent the results from an in vitro assay of the cellular response to IL-10 in (FIG. 10A) 130 donor monocyte/macrophage samples where the level of TNF-α secretion from monocytes/macrophages is measured in response to IL-10; and (FIG. 10B) 129 donor CD8+ T cells samples where the level of IFN-γ is measured in response to IL-10.

FIG. 11 represents the repeat assessment of monocyte/macrophages response to IL-10 in three donors (donors 240, 307, and 50720).

FIG. 12 represents the repeat assessment of CD8+ T cell stimulatory response to IL-10 in three donors (donors 240, 307, and 50720).

FIG. 13 represents the results from the gene expression profile analysis demonstrating that there are clusters of genes expressed in CD8+ T cells in low, medium and high responders.

FIG. 14 is a schematic representation of the screening process for determine the gene expression profile and/or SNP and/or INDEL profile in CD8+ T cells for oncology.

FIG. 15 represents the results demonstrating that IL-10 and IL-10 based agent are capable of suppressing pro-inflammatory stimulation (by LPS) of bulk cultures comprising monocytes/macrophages.

FIG. 16 represents the results demonstrating that in the absence of purification, anti-CD3 is ineffective in stimulating CD8+ T cells, demonstrating that CD8+ T cells must be purified to assess response to IL-10 or IL-10 based agent.

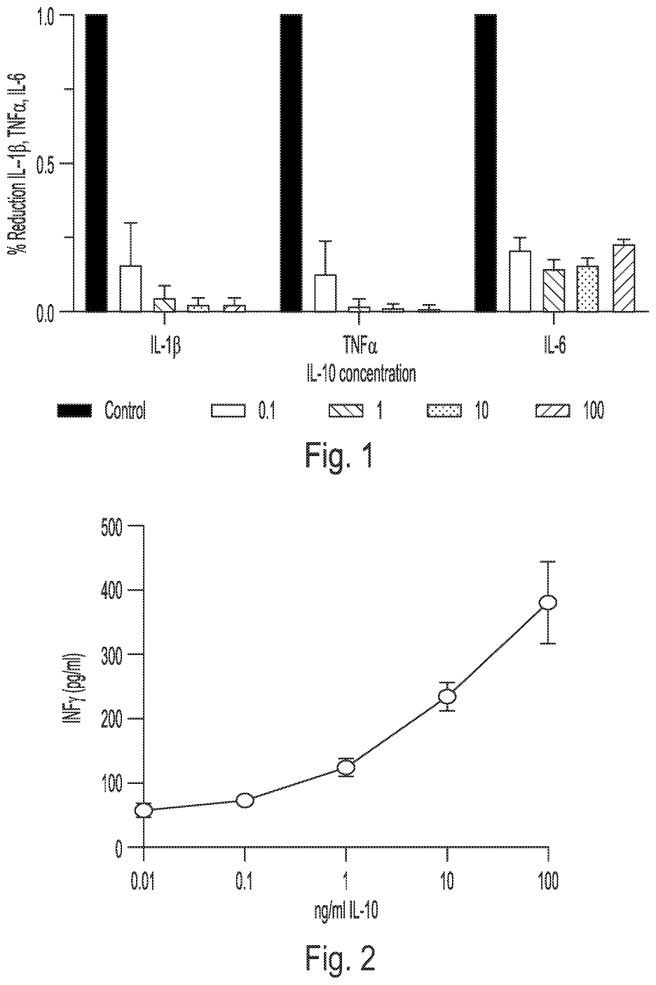

### DETAILED DESCRIPTION OF EMBODIMENTS

It should be understood that the products (such as the gene expression profile, genetic markers or the SNP and/or INDEL sequences and profiles), methods of determining, genotyping, and identifying, and methods of treating are not limited to the particular methods, procedures, assays, techniques, cell types, and reagents described herein. The terms used to describe the particular embodiments in the present application should not be construed to limit the scope of the present application as set forth in the appended claims. Any publications, patents and published patent applications referenced in this application are hereby incorporated by reference in their entirety.

Unless otherwise indicated, the embodiments described herein employ conventional methods and techniques of molecular biology, biochemistry, pharmacology, chemistry, and immunology, well known to those skilled in the art. Many of the general techniques for designing and fabricating the IL-10 variants, including but not limited to human, CMV and/or EBV forms of IL-10, as well as the assays for testing the IL-10 variants, are well known methods that are readily available and detailed in the art. See, e.g., Sambrook, et al., Molecular Cloning: A Laboratory Manual (2^{nd} Edition, 1989); Methods In Enzymology (S. Colowick and N. Kaplan eds., Academic Press, Inc.); Handbook of Experimental Immunology, Vols. I-IV (D. M. Weir and C. C. Blackwell eds., Blackwell Scientific Publications); A. L. Lehninger, Biochemistry (Worth Publishers, Inc., current addition); see also, U.S. Pat. No. 10,858,412 for the design of IL-10 fusion proteins comprising monomers of IL-10 and/or IL-10 variants and derivatives thereof. N-terminal aldehyde based PEGylation chemistry is also well known in the art.

Until the present application, it was not clearly understood why only a small percentage of Crohn's patients responded to reconstitutional IL-10 therapy. In a 2000 study, the administration of recombinant human IL-10 (5 microgram/kg) induced approximately 25% remission in subjects with active Crohn's disease. Fedorak, 2000. Another study found that recombinant human IL-10 reduced the secretion levels of TNF-α by monocytes/macrophages and induced T-cell production of IFN-γ, especially at higher IL-10 doses, in only a portion of the population. Tilg, 2002. The inventor of the present application attributes IL-10 response in these patients to a genetic predisposition whereby active remission is based on the possession of certain and specific gene expression profiles and/or SNP and/or INDEL sequences.

Similarly, it is unclear why approximately 25% of cancer patients respond to IL-10 therapy with demonstrable tumor reductions in a 2016 to 2018 study. Naing A., 2018; Naing A., 2016. The inventor of the present application attributes IL-10 response in these patients to a genetic predisposition whereby active remission is based on the possession of certain and specific gene expression profiles and/or SNP and/or INDEL sequences.

Accordingly, the inventor has unexpectedly found that to optimize treatment of certain inflammatory diseases or disorders, such as, for example, Crohn's disease, inflammatory bowel disease (IBD) or ulcerative colitis; and/or to treat cancer or oncology patients, screening an individual that possesses the correct phenotypic traits, such as low producers of IL-10 but having an increased response rate to IL-10 would be the most idea subclass of inflammatory patients to treat.

Similarly, the inventor has unexpectedly found that to optimize treatment of certain oncology disorders, screening an individual that possesses the correct phenotypic traits, such as increased CD8+ T cell production of IFN-γ in response to IL-10 would be the most ideal subclass of oncology patients to treat.

Accordingly, the present application relates to gene expression profiles and/or SNP and/or INDEL profiles associated with an increased likelihood of responding to IL-10, IL-10 derivative, or IL-10 based agent treatment or combination treatment including IL-10, IL-10 derivative, or IL-10 based agent with a secondary cytokine. Other aspects of the application include methods of determining or assaying for gene expression profiles and/or SNP and/or INDEL profiles in both healthy subjects and in diseased subjects and comparing the profiles of each to determine the optimal profile that results in the most receptive subjects to IL-10, IL-10 derivative, or IL-10 based agent treatment, or a combination of IL-10, IL-10 derivative, or IL-10 based agent with a second cytokine. An aspect of the invention also includes treating a diseased subject who has been identified as possessing the gene expression profile and/or SNP and/or INDEL profile that is most indicative of a positive response to IL-10, IL-10 derivative, or IL-10 based agent treatment, by administering reconstitutional IL-10, IL-10 derivative, or IL-10 based agent.

As used herein in describing the various embodiments, the singular forms “a”, “an” and “the” include plural referents unless the content clearly dictates otherwise.

The term “about” refers to a deviance of between 0.0001-25% from the indicated number or range of numbers. In one embodiment, the term “about”, refers to a deviance of between 1-10% from the indicated number or range of numbers. In one embodiment, the term “about”, refers to a deviance of up to 25% from the indicated number or range of numbers. In a more specific embodiment, the term “about” refers to a difference of 1-25% in terms of nucleotide sequence homology or amino acid sequence homology when compared to a wild-type sequence.

A “protein”, “polypeptide” or “peptide” is used interchangeably to refer to a product expressed by a gene.

A “recombinant protein” is a polypeptide produced by recombinant DNA techniques, where DNA encoding the protein is inserted into a suitable expression vector that is then used to transfect, infect, or transduce a host cell to produce the heterologous protein.

The term “genotype” refers to the specific allelic composition of an entire cell or a certain gene, whereas the term “phenotype” refers to the detectable outward expression of a specific genotype.

A “polymorphism” is the coexistence of more than one form of a gene or portion thereof. A portion of a gene of which there are at least two different forms or two different nucleotide sequences, is referred to as a “polymorphic region of a gene.” A polymorphic region can be a single nucleotide, which differs in different alleles.

The term “interleukin-10” or “IL-10” refers to a protein comprising two subunits non-covalently joined to form a homodimer. As used herein, unless otherwise indicated “interleukin-10” and “IL-10” can refer to human IL-10 (“hIL-10”; Genbank Accession No. NP_000563; or U.S. Pat. No. 6,217,857) protein (SEQ ID No: 1) or nucleic acid (SEQ ID No: 2); mouse IL-10 (“mIL-10”; Genbank Accession No: M37897; or U.S. Pat. No. 6,217,857) protein (SEQ ID No: 7) or nucleic acid (SEQ ID No: 8); or viral IL-10, (“vIL-10”). Viral IL-10 homologs may be derived from EBV or CMV (Genbank Accession Nos. NC_007605 and DQ367962, respectively). The term EBV-IL10 refers to the EBV homolog of IL-10 protein (SEQ ID No: 3) or the nucleic acid (SEQ ID No: 4). The term CMV-IL10 refers to the CMV homolog of IL-10 protein (SEQ ID No: 5) or the nucleic acid (SEQ ID No: 6).

The terms “wild-type,” “wt” and “native” are used interchangeably herein to refer to the sequence of the protein (e.g. IL-10 or EBV-IL10) as commonly found in nature in the species of origin of the specific IL-10 in question. For example, the term “wild-type” or “native” EBV-IL10 would thus correspond to an amino acid sequence that is most commonly found in nature.

The term “derive,” “derived,” “derive from,” or “derived from,” is used herein to identify the original source of a molecule, such as a viral form of IL-10 molecule, but is not meant to limit the method in which the molecule is prepare, manufactured, fabricated, or made. This would include methods, such as but not limited to, chemical or recombinant means.

The term “derivative” is intended to include any suitable modification of the reference molecule of interest or of an analog thereof, such as sulfation, acetylation, glycosylation, phosphorylation, polymer conjugation, hesylation, or other addition of foreign moieties, so long as the desired biological activity (e.g., anti-inflammation and/or no T cell stimulation) of the reference molecule or the variant is retained.

The terms “variant,” “analog” and “mutein” refer to biologically active derivatives of the reference molecule, that retain a desired activity, such as, for example, anti-inflammatory activity. Generally, the terms “variant,” “variants,” “analog” and “mutein” as it relates to a polypeptide refers to a compound or compounds having a native polypeptide sequence and structure with one or more amino acid additions, substitutions (that are conservative in nature), and/or deletions, relative to the native molecule.

A “variant” may include modifications (e.g., additions, substitutions, and/or deletions) that do not destroy the biological activity of the reference molecule. These variants may be “homologous” to the reference molecule as defined below. In general, the amino acid sequences of such analogs will have a high degree of sequence homology to the reference sequence, e.g., amino acid sequence homology of more than 50%, generally more than 60%-70%, even more particularly 80%-85% or more, such as at least 90%-95% or more, when the two sequences are aligned. Often, the analogs will include the same number of amino acids but will include substitutions. The variant will retain biological activity that is enhanced, diminished or substantially the same as the native molecule. Specifically, the term “variant” IL-10 molecule, which is interchangeable with the terms “engineered” IL-10 molecule or IL-10 variant molecule or IL-10 variant, refers to an IL-10 molecule or protein that includes one or both modifications to the IL-10 receptor binding domain(s) and/or to the regions responsible for forming an inter-domain angle in the IL-10 molecule or protein.

An “IL-10 agent” or “IL-10 based agent” is intended to be a collective term used to describe a molecule that differs from the wild type form of IL-10. The IL-10 agent will retain certain biological aspects of a fully functioning dimer of IL-10. Thus, the IL-10 agent includes any non-native form of IL-10 including those molecules that comprises a part, portion, fragment, and/or variant of IL-10. An IL-10 agent also includes an engineered fusion protein or chimeric protein wherein native monomers of IL-10 or variant forms of IL-10 (including Epstein Barr viral forms) are conjugated to antibody fragment as described in U.S. Pat. No. 10,858,412.

An “analog” or “analogs” may include substitutions that are conservative in nature. For example, conservative substitutions might include in kind type substitutions such as, but not limited to (1) an acidic substitution between aspartate and glutamate; (2) a basic substitution between any one of lysine, arginine, or histidine; (3) a non-polar substitution between any one of alanine, valine, leucine, isoleucine, proline, phenylalanine, methionine, or tryptophan; and (4) a uncharged polar substitution between any one of glycine, asparagine, glutamine, cysteine, serine threonine, or tyrosine. Phenylalanine, tryptophan, and tyrosine are sometimes classified as aromatic amino acids. It is also possible that an isolated replacement of leucine with isoleucine or valine, an aspartate with a glutamate, a threonine with a serine, or a similar conservative replacement of an amino acid with a structurally related amino acid may be made so long as the desired and specific biological activity is intact. For example, the polypeptide of interest may include up to about 1-10 conservative or non-conservative amino acid substitutions, or even up to about 15-25 conservative or non-conservative amino acid substitutions, or any integer between 1-50, so long as the desired function of the molecule remains intact. One of skill in the art may readily determine regions of the molecule of interest that can tolerate change well known in the art.

The term “homology,” “homologous” or “substantially homologous” refers to the percent identity between at least two polynucleotide sequences or at least two polypeptide sequences. Sequences are homologous to each other when the sequences exhibit at least about 50%, preferably at least about 75%, more preferably at least about 80%-85%, preferably at least about 90%, and most preferably at least about 95%-98% sequence identity over a defined length of the molecules.

The term “sequence identity” refers to an exact nucleotide-by-nucleotide or amino acid-by-amino acid correspondence. Percent identity can be determined using a variety of methods including but not limited to a direct comparison of the sequence information between two molecules (the reference sequence and a sequence with unknown % identity to the reference sequence) by aligning the sequences, counting the exact number of matches between the two aligned sequences, dividing by the length of the reference sequence, and multiplying the result by 100. Readily available computer programs can be used to aid in the identification of percent identity.

The term “fragment” is intended to include a portion of a molecule of the full-length amino acid or polynucleotide sequence and/or structure. A fragment of a polypeptide may include, for example, a C-terminal deletion, an N-terminal deletion, and/or an internal deletion of the native polypeptide. Active or functional fragments of a particular protein will generally include at least about 5-10 contiguous amino acid residues of the full-length molecule, preferably at least about 15-25 contiguous amino acid residues of the full-length molecule, and most preferably at least about 20-50 or more contiguous amino acid residues of the full-length molecule, or any integer between 5 amino acids and the full-length sequence, provided that the fragment in question retains biological activity, such as anti-inflammatory activity.

The term “substantially purified” generally refers to isolation of a substance such that the substance comprises the majority percent of the sample in which it resides. A substantially purified component comprises 50%, preferably 80%-85%, more preferably 90-95% of the sample. Likewise the term “isolated” is meant, when referring to a polypeptide or a polynucleotide, that the indicated molecule is separate and discrete from the whole organism with which the molecule is found in nature or is present in the substantial absence of other biological macro-molecules of the same type.

The terms “subject,” “individual,” or “patient” are used interchangeably herein and refer to a vertebrate, preferably a mammal. Mammals include, but are not limited to, murine, rodent, simian, human, farm animals, sport animals, and certain pets.

The term “administering” includes routes of administration which allow the active ingredient of the invention to perform their intended function.

A “therapeutically effective amount” as it relates to, for example, administering the EBV-IL-10 variants described herein, refers to a sufficient amount of the EBV-IL10 variant to promote certain biological activities. These might include, for example, suppression of myeloid cell function, enhanced Kupffer cell activity, and/or lack of any effect on CD8+ T cells or enhanced CD8+ T-cell activity as well as blockade of mast cell upregulation of Fc receptor or prevention of degranulation. Thus, an “effective amount” will ameliorate or prevent a symptom or sign of the medical condition. Effective amount also means an amount sufficient to allow or facilitate diagnosis.

The term “treat” or “treatment” refers to a method of reducing the effects of a disease or condition. Treatment can also refer to a method of reducing the underlying cause of the disease or condition itself rather than just the symptoms. The treatment can be any reduction from native levels and can be, but is not limited to, the complete ablation of the disease, condition, or the symptoms of the disease or condition.

An IL-10 or IL-10 based agent (or derivatives and variants thereof) responsive gene expression profile and/or SNP and/or INDEL profile or an expression profile and/or genetic marker and/or SNP and/or INDEL profile indicative of responsiveness to IL-10 (or an IL-10 agent) is defined as a profile of one or more expressed proteins and/or genetic markers and/or SNP and/or INDEL sequences found in the genome of an individual who is more likely to respond to IL-10 or IL-10 agent treatment.

A “proinflammatory stimulus” is meant as an agent or condition that induces a proinflammatory response by inflammatory cells, such as macrophages. Proinflammatory response is characterized by secretion of one or more proinflammatory molecules, such as TNF-α, IL-1, IL-6, IL-12, IL-23. The proinflammatory stimulus may include agents such as bacteria or components thereof, including bacterial cell walls, such as lipopolysaccharide (LPS). The activation of an inflammatory cell, such as a monocyte/macrophage, by a proinflammatory stimulus results in a stimulated cell.

The term “bispecific molecule” is intended to include any agent, e.g., a protein, peptide, or protein or peptide complex, which has two different binding specificities. The term “multispecific molecule” or “heterospecific molecule” is intended to include any agent, e.g., a protein, peptide, or protein or peptide complex, which has more than two different binding specificities.

The term “response” implies any kind of improvement or positive response, either clinical or non-clinical such as, but not limited to, measurable reduction or treatment of a disease or disorder, such as inflammatory disease, reduction tumor size or evidence of disease or disease progression, complete response, partial response, stable disease, increase or elongation of progression free survival, increase or elongation of overall survival, or reduction in toxicity.

The term “likely to respond,” “increased likelihood of responding,” “higher likelihood” or “increased chance of responding” shall mean that the patient or subject is more likely than not to exhibit at least one measurable improvement or change, in terms of a quantifiable measurement of particular markers, proteins, cytokines, etc., as compared to similarly situated patient or subject who does not have any measurable improvement or change.

The term “high” Tumor Necrosis Factor-alpha (TNF-α) reduction or suppression or response or responsiveness in monocytes/macrophages in response to a pro inflammatory stimulus shall mean about 10 to 15% (preferably 12%) reduction in TNF-α secretion by monocytes/macrophages. The term “medium” or “intermediate” TNF-α reduction or suppression or response or responsiveness in monocytes/macrophages in response to a pro inflammatory stimulus shall mean about 20 to 30% (preferably 26%) reduction in TNF-α secretion by monocytes/macrophages. The term “low” TNF-α reduction or suppression or response or responsiveness in monocytes/macrophages in response to a pro inflammatory stimulus shall mean about 40 to 50% (preferably 44%) reduction in TNF-α secretion by monocytes/macrophages.

The term “high” induction or production or secretion of IL-10 in monocytes/macrophages in response to a pro-inflammatory stimulus shall mean about 1550 to 3000 picograms (pg)/ml (preferably 1658 to 2802 pg/ml) at 1 to 100 ng/ml of a pro-inflammatory stimulus (e.g., LPS) respectively, as measured by standard ELISA. The term “medium” or “intermediate” induction or production or secretion of IL-10 in monocytes/macrophages in response to a pro-inflammatory stimulus shall mean about 630 to 1500 pg/ml (preferably 630 to 1242 pg/ml) at 1 to 100 ng/ml of a pro-inflammatory stimulus (e.g., LPS) respectively, as measured by standard ELISA. The term “low” induction or production or secretion of IL-10 in monocytes/macrophages in response to a pro-inflammatory stimulus shall mean about 300 to 615 pg/ml (preferably 318 to 615 pg/ml) at 1 to 100 ng/ml of a pro-inflammatory stimulus (e.g., LPS) respectively, as measured by standard ELISA.

The term “high” Interferon-gamma (IFN-γ) level or secretion or induction or response by CD8+ T cells shall mean an IFN-γ level of about greater than or equal to 850 pg/ml (preferably 852 to 4277 pg/ml) at 1 to 100 ng/ml of IL-10, IL-10 based agent, or derivatives or variants thereof, stimulation respectively as measured by standard IFN-γ ELISA. The term “medium” or “intermediate” IFN-γ level or secretion or induction or response by CD8+ T cells shall mean an IFN-γ level of about greater than or equal to 350 to 849 pg/ml (preferably 368 to 842 pg/ml) at 1 to 100 ng/ml IL-10, IL-10 based agent, or derivatives or variants thereof, stimulation respectively as measured by standard IFN-γ ELISA. The term “low” IFN-γ level or secretion or induction by CD8+ T cells shall mean an IFN-γ level of about greater than or equal to 0 to 349 pg/ml (preferably 0 to 375 pg/ml) at 1 to 100 ng/ml IL-10, IL-10 based agent, or derivatives or variants thereof, stimulation respectively as measured by standard IFN-γ ELISA

As noted above, the inventor discovered certain gene expression profiles and/or SNPs and/or INDELs that correlates with a higher likelihood of responding to IL-10 or IL-10 based agent treatments. The gene expression profile and/or SNP and/or INDEL profile(s) is associated with a population of individuals who are phenotypically lower producers of IL-10 and yet have the capacity to respond to suppression or stimulation from IL-10. This includes individuals who phenotypically possess monocytes/macrophages that have (1) low production of IL-10 and (2) high capacity to suppress TNF-α (upon stimulation by a pro-inflammatory stimulus); or phenotypically possess CD8+ T cells that, when stimulated by IL-10 (or an IL-10 based agent) to induce high levels of IFN-γ. Thus, one embodiment of the present application includes a method for selecting or screening for patients more receptive to IL-10 or IL-10 based agent treatment comprising the examination of gene expression profiles and/or SNP and/or INDEL profiles associated with a higher likelihood of responding to the IL-10 or IL-10 based agent. In another embodiment, the present application relates to a kit comprising primers and probes that are useful in determining the presence of the gene expression profiles and/or genetic SNP and/or INDEL profile.

In order to determine the requisite gene expression profile and/or SNP and/or INDEL profile associated with an increased likelihood of response to IL-10 or IL-10 based agent, the inventor devised an in vitro model system to measure IL-10 responsiveness. The process of assessing the genetic component in this model system, requires a two-tiered approach of (1) gathering response data from a cellular assay followed by (2) a genetic analysis of certain phenotypic traits exhibited in the cellular assay.

### Cellular Assay: Inflammatory Disease Responsiveness

The cellular assay for inflammatory disease involves isolating monocytes/macrophages from both healthy and diseased donors to determine responder profiles in inflammatory disease. The monocytes/macrophages from the donors are stimulated and assayed for (1) production or secretion of IL-10 and (2) response to IL-10 measured by the level of TNF-α. For the production, secretion, or induction of IL-10, a pro-inflammatory stimulus, such as LPS, is administered to the donor monocytes/macrophages and the amount of IL-10 produced, secreted, or induced is measured. Typically, monocytes/macrophages are induced to produce IL-10 in response to a pro-inflammatory stimulus, such as LPS. Stimulating monocytes/macrophages with a pro-inflammatory stimulus, such as LPS, resulted in three different donor populations of low, medium, and high producers or secretors of IL-10. Likewise, stimulation with a pro-inflammatory cytokine also exhibit differential suppressive responses to IL-10 as measured by the level of TNF-α and resulted in high, medium, and low reduction in TNF-α secretion (i.e., high, medium, and low responders to IL-10 as measured by the level of TNF-α secretion). These various phenotypic traits (i.e., high, medium, low production or secretion of IL-10 and/or high medium, low reducers of TNF-α secretion) are sorted and grouped for subsequent genetic analysis.

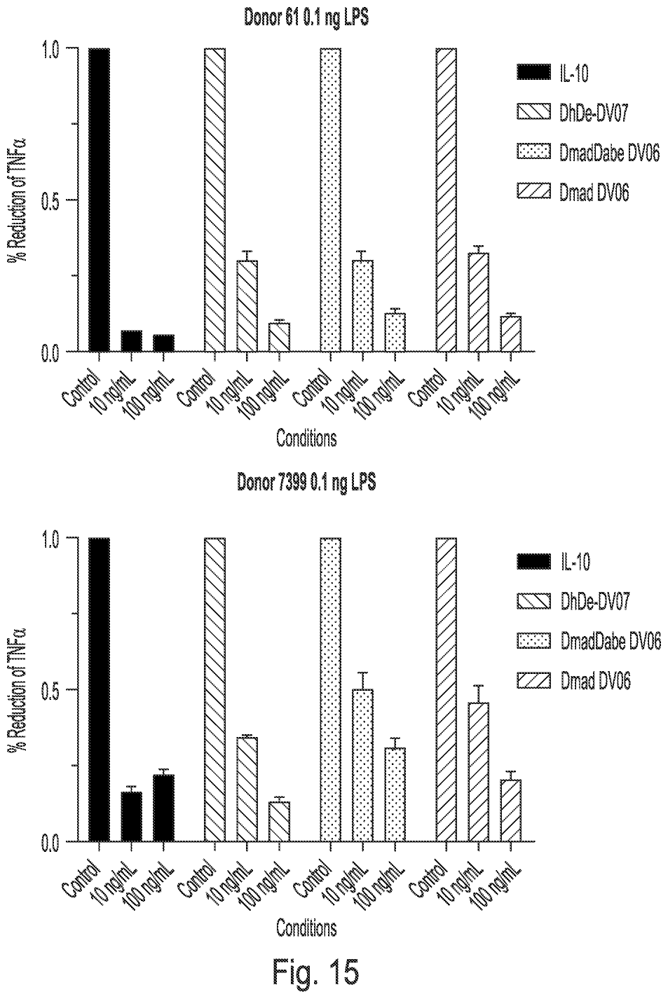

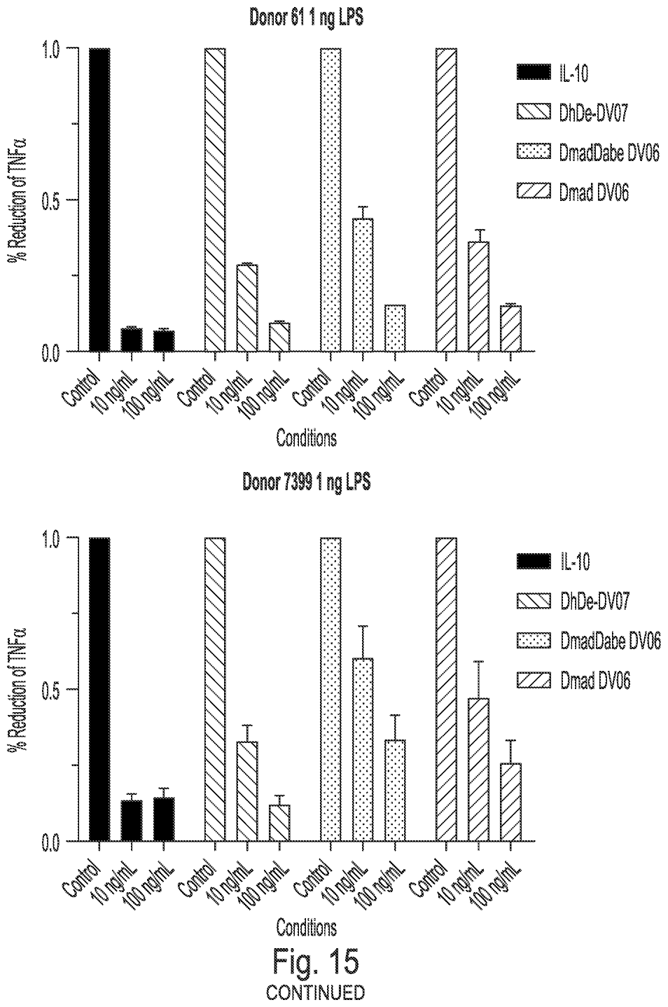

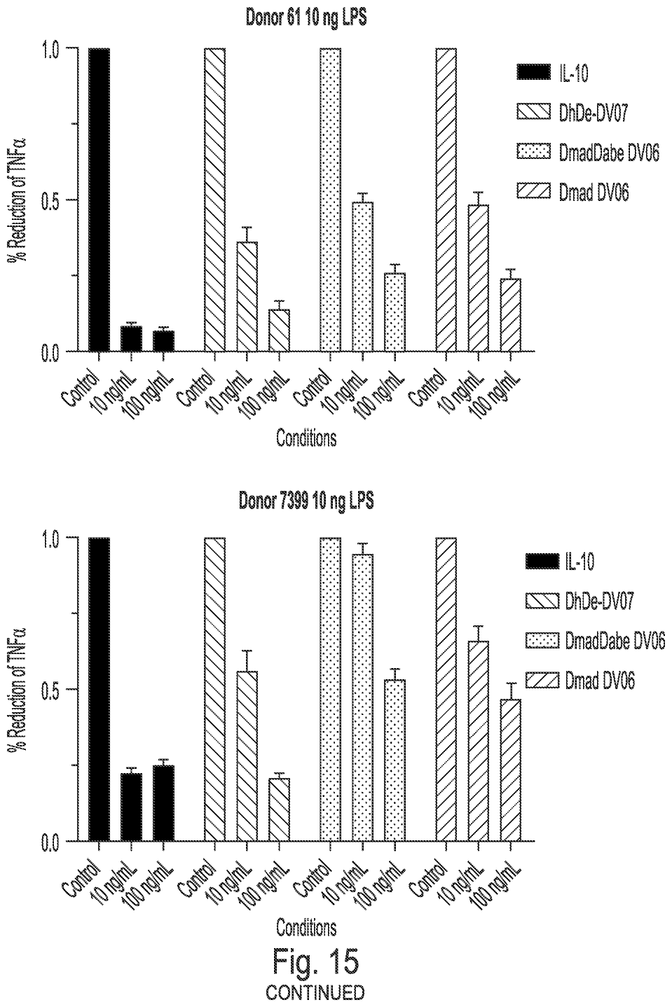

In a separate embodiment of this assay, bulk peripheral blood mononuclear cells (PBMC) (i.e., without purification of individual cells) are exposed to IL-10 or IL-10 based agent, after which LPS is added to the culture for 12-24 hours. The level of TNF-α found in the supernatant is quantified. It was surprisingly found that purification of monocytes/macrophages from bulk PBMC cultures is not required prior to stimulation by a pro-inflammatory agent for the assessment of TNF-α quantification. FIG. 15.

### Cellular Assay: Oncology Responsiveness

The cellular assay for oncology involves the isolation of CD8+ T cells from both health and disease donors and assessing the cells ability to respond to IL-10 or IL-10 based agent stimulation. Typically, IL-10 or IL-10 based agent administration to CD8+ T cells results in the stimulation of the CD8+ T cells to produce, secrete or induce IFN-γ secretion. The cellular assay involves isolation of CD8+ T cells from PBMC. The CD8+ T cells from donors are activated/stimulated by anti-CD3/anti-CD28 and then exposed to an IL-10 or IL-10 based agent. After exposure to an IL-10 or IL-10 based agent, the cells are washed and exposed to soluble anti-CD3 for 4 hours, after which IFN-γ present in the supernatant is quantified.

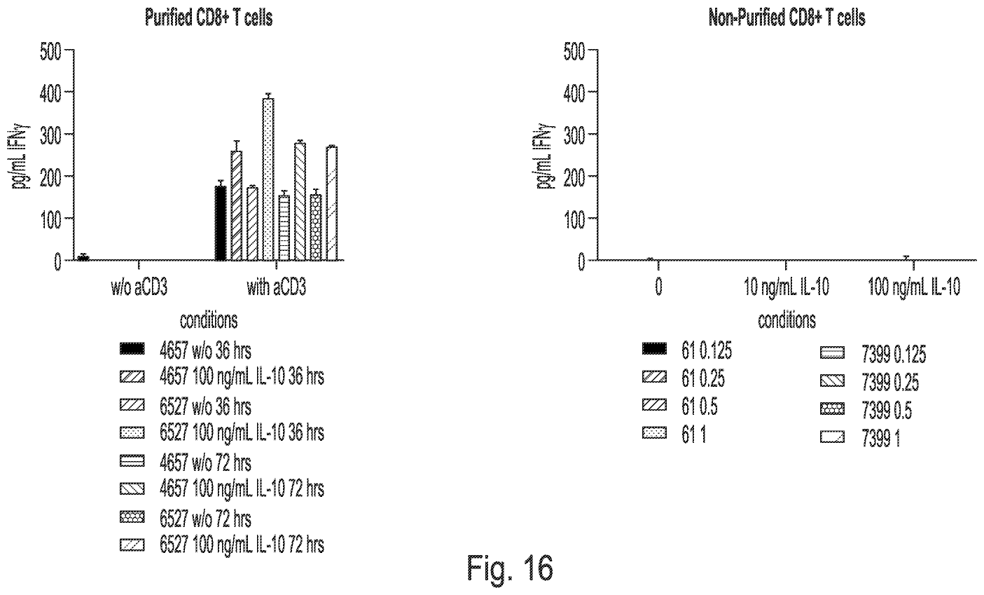

In a separate embodiment, bulk PBMC are exposed to an IL-10 or IL-10 based agent. After exposure to an IL-10 or IL-10 based agent, the CD8+ T cells are isolated and exposed to soluble anti-CD3 for 4 hours, after which IFN-γ present in the supernatant is quantified. It was surprisingly found that assessment of CD8+ T cell response to IL-10 or IL-10 based agent, in this model cellular system, requires isolation prior to activation with anti-CD3/anti-CD28. FIG. 16.

It was also surprisingly found that stimulating CD8+ T cells with IL-10 or IL-10 based agent resulted in three different donor populations of high, medium, and low responders to IL-10. These various phenotypic traits (i.e., high, medium, low responders to IL-10) are sorted and grouped for subsequent genetic analysis

### Genetic Analysis of Certain Phenotypic Responses

To determine the underlying genetic components associated with the different phenotypic responses in monocytes/macrophages and in CD8+ T cells, an examination of (1) the differences in gene expression signatures between the high, medium, and low populations (both for IL-10 production or secretion and response to an IL-10 or IL-10 based agent via the secretion of TNF-α in response to a pro-inflammatory stimulus in macrophages/monocytes, as well as the response to an IL-10 or IL-10 based agent via the secretion or induction of IFN-γ in CD8+ T cells), and (2) the underlying or background genetic mutations in genes, are performed. The data gleaned from both the gene expression signatures and the underlying background genetic mutational data generates a blended data set that permits the identification of common pathways associated with each of the high, medium, and low populations in monocytes/macrophages and in CD8+ T cells. Furthermore, an aggregated analysis of various patient samples having different phenotypic traits (i.e., high, medium, and low secretors of TNF-α and IL-10 production/secretors in response to a pro-inflammatory stimulus in monocytes/macrophages as well as responders to IL-10 or IL-10 based agents in CD8+ T cells) establishes a standard gene expression signatures and mutational frequency profile from which to compare whether a sample falls into the high, medium, and low populations.

To determine differentials in gene expression signatures, a comparison of the gene expression signatures between the three phenotypic responses in monocytes/macrophages (i.e., high, medium, and low populations for IL-10 production or secretion and high, medium, and low response to an IL-10 or IL-10 based agent via the secretion of TNF-α in response to a pro-inflammatory stimulus) and/or in CD8+ T cells (i.e., high, medium, and low response to an IL-10 or IL-10 based agent via the secretion or induction of IFN-γ) is/are performed. Those genes having a differential expression between high and low, high and medium, and/or medium and low populations, whereby the differential gene expression is five, four, three, or two fold different, will be considered genes relevant for identifying, screening, or classifying a patient that is responsive or non-responsive to IL-10 or IL-10 based agent treatment. Preferably, the gene expression signature associated with low production or secretion of IL-10 along with high reduction in TNF-α secretion in monocytes/macrophages (i.e., high response to IL-10 or IL-10 based agent) following a pro-inflammatory stimulus will be indicative of a patient that will be responsive to IL-10 or IL-10 based agent treatment for inflammatory disease. Also preferable, the gene expression signature associated with high response to IL-10 or IL-10 based agent via the secretion or induction of IFN-γ in CD8+ T cells will be indicative of a patient that will be responsive to IL-10 or IL-10 based agent treatment for oncology. Tables 3-8 provide a listing of genes expression profiles that are associated with CD8+ T cells that are high responders to IL-10 or IL-10 based agents (Table 3), low responders to IL-10 or IL-10 based agents (Table 4), as measured by IFN-γ production or secretion; high IL-10 production or secretion (Table 5) or low IL-10 production or secretion (Table 6), high IL-10 responsiveness as measured by TNF-α production (Table 7) or low IL-10 responsiveness (Table 8) as measured by TNF-α production, in response to a pro-inflammatory stimulus in monocytes/macrophages.

Along with the gene expression signature data, the underlying genetic information, which examines the gene mutational frequency between the three phenotypic responses in monocytes/macrophages (i.e., high, medium, and low populations for IL-10 production or secretion and high, medium, and low response to an IL-10 or IL-10 based agent via the secretion of TNF-α in response to a pro-inflammatory stimulus) and/or in CD8+ T cells (i.e., high, medium, and low response to an IL-10 or IL-10 based agent via the secretion or induction of IFN-γ), is/are performed. Those genes that are most differentially mutated between the high and low, high and medium, and/or medium and low populations are examined for SNP and/or INDEL profiles. Genes having mutational frequencies that are five, four, three, or two fold different between the high and low, high and medium, and/or medium and low populations, will be considered relevant genes for identifying, screening, or classifying a patient that is responsive or non-responsive to IL-10 or IL-10 based agent treatment. Tables 9-10 provide SNP and/or INDEL gene mutational frequencies associated with CD8+ T cells that are high responders to IL-10 or IL-10 based agents (Table 9), low responders to IL-10 or IL-10 based agents (Table 10), as measured by IFN-γ production or secretion; high IL-10 production or secretion (Table 11) or low IL-10 production or secretion (Table 12), high IL-10 responsiveness as measured by TNF-α production (Table 13) or low IL-10 responsiveness (Table 14) as measured by TNF-α production, in response to a pro-inflammatory stimulus in monocytes/macrophages.

Finally, the data obtained from both the gene expression signatures and mutational frequency information (collectively referred to as a “hit count”) is blended together and referenced against known and publicly available molecular pathway databases (e.g., Reactome Pathway database) to determine common molecular pathways in which the differential gene expression signatures and the genes having the highest mutational frequency are involved. Commonalities in pathways are determined by a statistical p value. Along with the gene expression signatures, tables 3-8 also provides the molecular pathways and the associated gene expression signatures associated with the molecular pathways.

The process of determining the differential gene expression and screening for gene mutational frequency is conventional and may be accomplished by any means generally known in the art. This may include differential display, direct sequencing of the gene, entire genome sequencing, and/or characterization of an expression product of a gene, such as mRNA, peptide, or proteins.

The genetic component may include, for example genomic DNA, amplified genomic DNA, cDNA, amplified cDNA, RNA, amplified RNA, or other genetic components known to those of skill in the art. The molecular pathway and corresponding gene expression signature are as follows: CD8+ T cells that are high responders to IL-10 or IL-10 based agents (Table 3) and low responders to IL-10 or IL-10 based agents (Table 4), as measured by IFN-γ production or secretion; high IL-10 production or secretion (Table 5) or low IL-10 production or secretion (Table 6), high IL-10 responsiveness as measured by TNF-α production (Table 7) or low IL-10 responsiveness (Table 8) as measured by TNF-α production, in response to a pro-inflammatory stimulus in monocytes/macrophages.

A combination of the gene expression signatures, gene mutational frequencies, and blended common molecular pathways data for each of the high, medium, and low populations is used to determine a standard from which patients will be categorized to determine responsiveness to IL-10 or IL-10 based agent treatment.

The gene mutational frequency may be any number of differences at the nucleotide level that differs from native or wild-type or generally accepted reference sequences, polynucleotide sequences. Preferably the gene mutation is/are a SNP and/or INDEL but the gene mutation may also include simple sequence repeats (SSRs), restriction fragment length polymorphisms (RFLPs), and amplified fragment length polymorphisms (AFLPs), Differential gene expression level is determined by hit count analysis of mRNA or qPCR or mRNA profiles. The genetic marker may include variability associated with insertions, deletions, duplications, repetitive elements, point mutations, recombination events, or the presence and sequence of transposable elements. Molecular markers can be derived from genomic or expressed nucleic acids (e.g., ESTs) and can also refer to nucleic acids used as probes or primer pairs capable of amplifying sequence fragments via the use of PCR-based methods.

Using the combined gene expression signatures and/or SNP and/or INDEL genetic frequency data, genetic profiles are determined for those subjects that are more likely to respond to IL-10 or IL-10 based agent response. For treatment of inflammatory disease, the gene expression signatures, and genetic frequency data associated with both low production or secretion of IL-10 and high responsiveness to IL-10 or IL-10 based agent having the most significant reduction of TNF-α secretion will be the standards from which to determine patient most receptive to IL-10 or IL-10 based agent anti-inflammatory therapy. For treatment of oncology, the gene expression signatures, and genetic frequency data associated with high responsiveness to IL-10 or IL-10 based agent having the most significant production of IFN-γ secretion will be the standards from which to determine patient most receptive to IL-10 or IL-10 based agent oncology therapy.

In an embodiment, the application relates to a method of determining the likelihood that a patient with an inflammatory disease will respond to IL-10 or IL-10 based agent comprising isolating and analyzing a genetic component from a subject for the presence of a gene expression profile (e.g., assessing mRNA levels) and/or SNP and/or INDEL profile or signature linked to a phenotypic responsiveness to IL-10 or IL-10 based agent treatment. In a preferred embodiments, the gene expression data relates to a molecular pathway and gene expression profile data found in tables 3-8.

In another embodiment, the application relates to a method of screening a patient for responsiveness to IL-10 or IL-10 agent based treatment comprising analyzing a genetic component from the patient for the presence of a SNP and/or INDEL profile linked to a phenotypic trait where inflammatory cells have both low induction of IL-10 and high responsiveness to IL-10 through increased reduction of TNF-α in monocytes/macrophages or significant increases in IFN-γ secretion by IL-10 or IL-10 agent exposed CD8+ T cells.

An anti-inflammatory cell may include any cell associated with the inflammatory response. These include, without limitation, monocytes, macrophages, dendritic cells, granulocytes, neutrophils, eosinophils, lymphocytes, plasma cells, and histiocytes. Preferably, the anti-inflammatory cell is a monocyte/macrophage.

An anti-cancer cell may then include any cell associated with targeting and destroying tumor cells. These include, without limitation, monocytes, macrophages, dendritic cells, CD4+ T cells, CD8+ T cells and NK cells. Preferably, the anti-cancer cell is a CD8+ T cell.

In one embodiment of the application, subjects with inflammatory disease are screened, selected, and administered an IL-10 or IL-10 based agent treatment. The differential response of inflammatory patients to IL-10 treatment can improved by specifically selecting those patients who exhibit the presently described expression profile and/or genetic marker and/or SNP and/or INDEL profile. Prior to the administration of an IL-10 or IL-10 based agent treatment, a selection of patients who are determined to be receptive to IL-10 will improve the chances of success. This is applicable to both oncology and IBD. Accordingly, when coupled with the method of screening described herein, patients determined to possess the expression profiles and/or genetic markers and/or SNP and/or INDEL profile correlative to receptive IL-10 response will be administered IL-10 or an IL-10 based agent. When an expression profile and/or genetic marker or is utilized for selecting a subject for a treatment described herein, the genetic marker or SNP and/or INDEL is measured before and/or during treatment, and the values obtained are used by a clinician to assess probable or likely success, failure, receptive to treatment, not receptive, or continued treatment.

The IL-10, a composition, or a formulation thereof may include human or viral forms. In an aspect, the IL-10 may be any of SEQ ID Nos:1-8. The IL-10 may be further amended to include modifications such as sulfation, acetylation, glycosylation, phosphorylation, polymer conjugation, hesylation, or other addition of foreign moieties, so long as the desired biological activity (e.g., anti-inflammation and/or limited to no T cell stimulation for IBD, or potent CD8+ T cell stimulation for oncology) of the reference molecule or the variant is retained. The IL-10 may also be a derivative or variant, which refers to an IL-10 amino acid (or nucleic acid) sequence that differs from wild type IL-10 anywhere from 1-25% in sequence identity or homology. Thus, for example, an EBV IL-10 variant molecule is one that differs from wild-type EBV IL-10 by having one or more amino acid (or nucleotide sequence encoding the amino acid) additions, substitutions and/or deletions. Thus in one form, an EBV IL-10 variant is one that differs from the wild type sequence of SEQ ID No.:3 by having about 1% to 25% difference in sequence homology, which amounts to about 1-42 amino acid difference.

In another aspect, the IL-10 is a functional variant that includes modifications (e.g., additions, substitutions, and/or deletions) that do not destroy the biological activity of the reference molecule. These variants may be “homologous” to the reference molecule as defined below. In general, the amino acid sequences of such analogs will have a high degree of sequence homology to the reference sequence, e.g., amino acid sequence homology of more than 50%, generally more than 60%-70%, even more particularly 80%-85% or more, such as at least 90%-95% or more, when the two sequences are aligned. Often, the analogs will include the same number of amino acids but will include substitutions. The functional variant will retain biological activity that is enhanced, diminished or substantially the same as the native molecule. Specifically, the term “variant” IL-10 molecule, which is interchangeable with the terms “engineered” IL-10 molecule or IL-10 variant molecule or IL-10 variant, refers to an IL-10 molecule or protein that includes one or both modifications to the IL-10 receptor binding domain(s) and/or to the regions responsible for forming an inter-domain angle in the IL-10 molecule or protein. A variant IL-10 “fusion protein” or “diabody” or “fusion” generally refers to the formation of a fusion protein (or a fusion protein complex) comprising variant IL-10 (in either monomeric form or in homodimeric form) and at least one other protein. As used herein a variant IL-10 “or a fusion protein thereof” may be used throughout this description to describe such a variant IL-10 fusion protein.

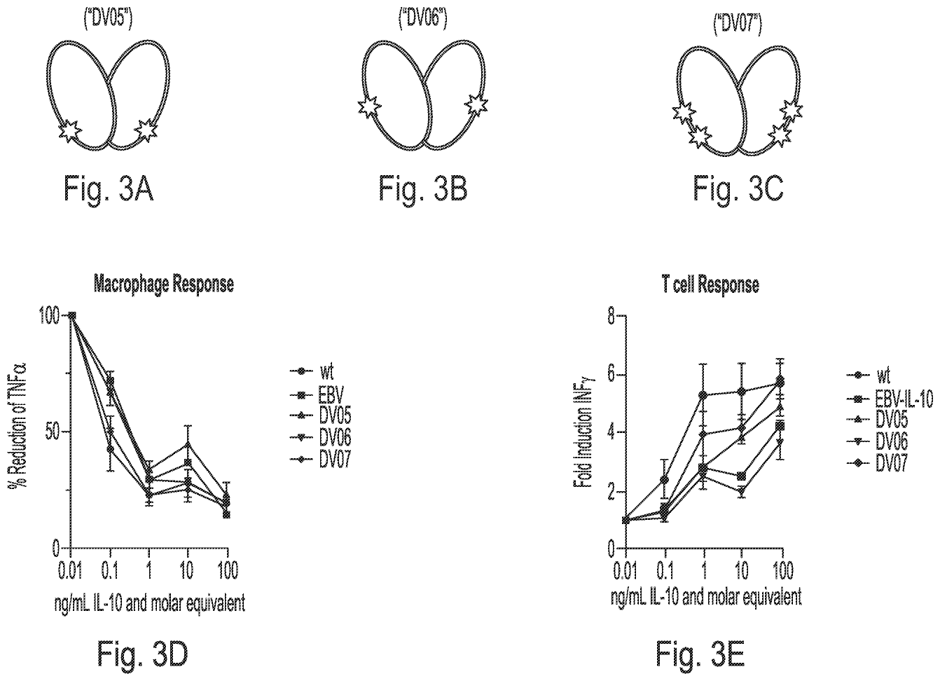

In a preferred embodiment, the IL-10 fusion protein, as described in U.S. Pat. No. 10,858,412 will include a specific modification to the IL-10 portion of the fusion protein. In a preferred embodiment, the IL-10 portion of the fusion protein will include an EBV IL-10 harboring a V31L substitution in SEQ ID No.:3. This modification has been shown to decrease macrophage response and is designated DV05. (FIG. 3A). In another preferred embodiment, the IL-10 portion of the fusion protein will include an EBV IL-10 harboring a A871 substitution in SEQ ID No.:3. This modification has been shown to decrease T-cell stimulation and is designated DV06. (FIG. 3B).

The fusion protein is an engineered protein comprising an IL-10 portion, a linker or spacer region, and VH and/or VL portion. The linker or spacer can be a random amino acid sequence (such as SSGGGGS (SEQ ID No.: 30, or GGGGSGGGGSGGGGS (SEQ ID No.: 31)), constant region of an antibody, a scFv, a diabody, or a minibody. The constant region can be derived from, but not limited to IgG1, IgG2, IgG3, IgG4, IgA, IgM, IgD, or IgE. The linker or spacer can be preferably the constant heavy (CH) region 1, CH2, or CH3. In a more preferred embodiment, the linker of spacer is a random amino acid sequence of SEQ ID Nos: 30 and/or 31.

The fusion protein may also include at least one monomer of IL-10 or IL-10 variant molecule conjugated at the fusion protein's N-terminal end, the C-terminal end, or both. In another embodiment, the fusion protein comprising IL-10 or IL-10 variant may also include at least one cytokine conjugated at the terminal end opposite of the IL-10 or variant IL-10 and includes IL-2, IL-7, IL-15, IL-26, IL-271L-28, IL-29, IL-10, IL-10 variant molecule, IFN-α, EPO, TGF-beta, b-FGF, EGF, PDGF, IL-4, IL-11, IL-13, or any combination thereof. In some preferred embodiments, the fusion protein comprises two monomeric forms of IL-10 or IL-10 variant molecules conjugated at the N-terminal end of the fusion protein and two IL-10 or IL-10 variant molecules conjugated at the C-terminal end of the fusion protein; the fusion protein comprises two monomeric forms of IL-10 or IL-10 variant molecules conjugated at the N-terminal end of the fusion protein and at least one IL-2 molecules (or other cytokine/growth factor) conjugated at the C-terminal end of the fusion protein; the fusion protein comprises two IL-10 or IL-10 variant molecules conjugated at the N-terminal end of the fusion protein and at least one IL-15 molecules conjugated at the C-terminal end of the fusion protein. In another embodiment, the C-terminal end of the fusion protein may at least two different cytokines selected from IL-10 or variant IL-10 and includes IL-2, IL-7, IL-15, IL-26, IL-27, IL-28, IL-29, IL-10, IL-10 variant molecule, IFN-α, EPO, TGF-beta, b-FGF, EGF, PDGF, IL-4, IL-11, IL-13, or any combination thereof.

In another embodiment, the fusion protein is fabricated using a scFv, a diabody, Fab, or any antibody fragment as the base scaffold onto which one monomer or two monomers of IL-10, one monomer or two monomers of a IL-10 variant molecule, IL-2, IL-7, IL-15, IL-26, IL-27, IL-28, IL-29, IFN-α, EPO, TGF-beta, basic-FGF, EGF, PDGF, IL-4, IL-11, or IL-13, or combinations thereof are conjugated. The scFv, diabody, minibody, Fab, or antibody fragment portion of the fusion protein may have a non-targeting or targeting functionality. The ability to target may be dictated by the presence of specific antigen binding modalities, such as the presence of CDR regions. These CDR regions are derived from monoclonal antibodies developed against specific target proteins, e.g. EGFR, VEGFR, FGFR, PDGFR, Her2Neu, GPC3, MAdCAM, VCAM, ICAM, that are grafted through established molecular biology techniques into the anti-Ebola VH VL that acts as a scaffolding system, by replacing the cognate CDR regions of the anti-Ebola VH VL.

In one particularly preferred embodiment, the fusion protein comprises at least one variable region, having a variable heavy chain (VH) and/or variable light chain (VL), linked to an IL-10 or IL-10 variant molecule. In this configuration, the fusion protein comprises an IL-10 monomer or variant IL-10 monomer linked to at least one variable region of an antibody. In one aspect, this fusion protein is a linear contiguous sequence comprising an IL-10 monomer or IL-10 monomer variant molecule linked to a VH, linked to a VL, linked to an IL-10 monomer. The variable region of the antibody can be a variable heavy (VH) chain region, a variable light (VL) chain region, or both. A first fusion protein comprises a protein sequence having a linear contiguous configuration such that an IL-10 monomer or a variant IL-10 monomer is conjugated to a variable region's (VH or VL or both) carboxy terminal end. A second fusion protein may comprise a protein sequence having a linear contiguous configuration such that an IL-10 monomer or a variant IL-10 monomer is linked to a variable region's (VH or VL or both) amino terminal end. A representative example of the first fusion protein described above may include the following configuration:

_{NH2}(Ab1VL)_{COOH}-(linker)-_{NH2}(monoIL10)_{COOH} a)

A representative example of the second fusion protein described above may include the following configuration:

_{NH2}(monoIL10)_{COOH}-(linker)-_{NH2}(Ab₁VH)_{COOH} b)

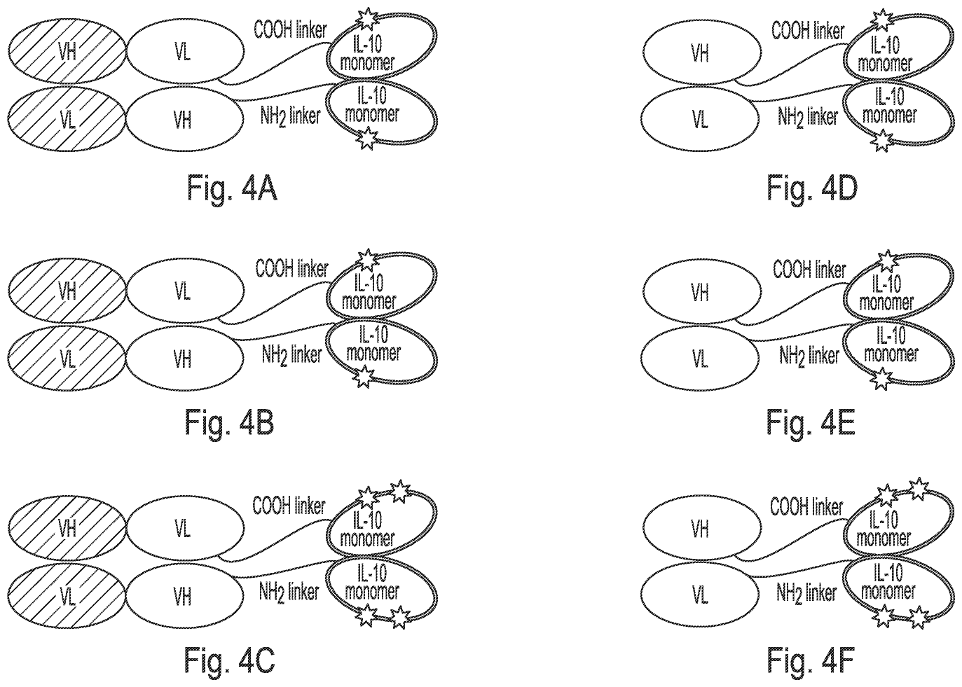

Together, the first (a) and second (b) fusion proteins form a functional protein complex in an anti-parallel manner, whereby the terminally linked monomers of IL-10 or variant IL-10 form a functional homodimer and the variable regions together are capable of forming a functional antigen binding site (“ABS”) (see e.g., FIGS. 4(a)-(f)).

In an alternative embodiment, the IL-10 monomer or variant IL-10 monomer may be conjugated to at least two variable regions from the same antibody or from two different antibodies. In this configuration, the at least two variable regions are a VH and VL. An example of such a configuration would include a first fusion protein with a linear contiguous protein sequence of a VH region of a first antibody linked at its carboxy terminal end to an amino terminal end of a VL region of the second antibody subsequently linked to the amino terminal end of a monomer of IL-10 or a monomer of an IL-10 variant molecule. An alternative configuration would include a second fusion protein with a linear contiguous protein sequence of a monomer IL-10 or a monomer of an IL-10 variant molecule linked at its carboxy terminal end to an amino terminal end of a VH region of the second antibody subsequently linked to an amino terminal end of a VL region of the first antibody. A representative example of the first fusion protein described above may include the following configuration:

_{NH2}(Ab₁₋VH)_{COOH}-(linker)-_{NH2}(Ab₂VL)_{COOH}-(linker)-_{NH2}(monoIL10)COOH a)

A representative example of the second fusion protein described above may include the following configuration:

_{NH2}(monoIL10)_{COOH}-(linker)-_{NH2}(Ab₂VH)_{COOH}-(linker)-_{NH2}(Ab₁₋VL)_{COOH} b)

Together, the first (a) and second (b) fusion proteins form a functional protein complex in an anti-parallel manner, whereby the terminally linked monomers of IL-10 or variant IL-10 form a functional homodimer and the variable regions together are capable of forming an ABS (see e.g., FIGS. 4(a)-(c)).

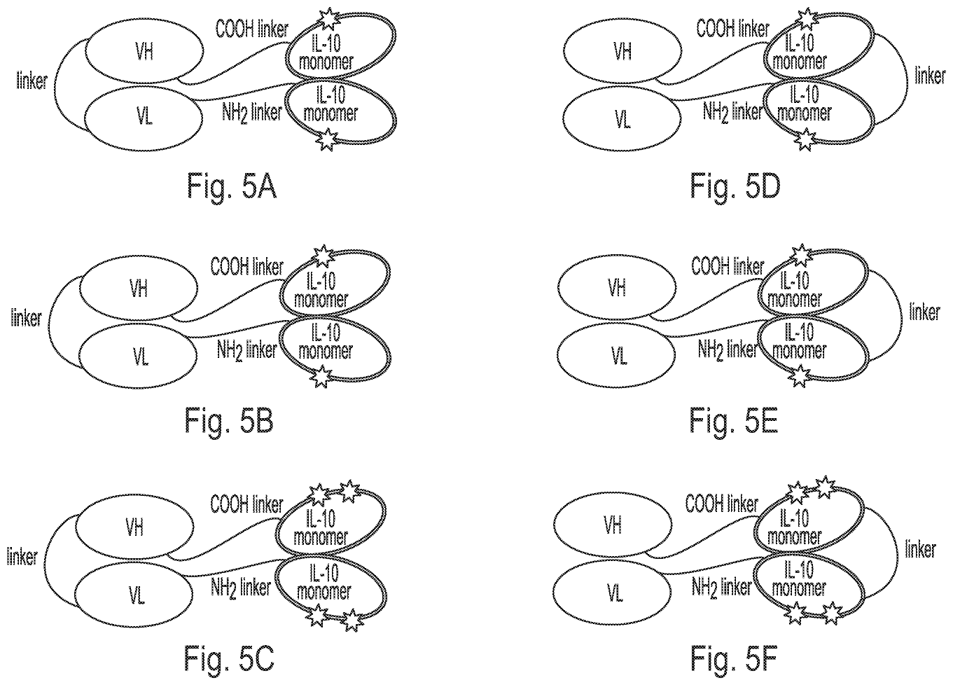

In yet another embodiment, the fusion protein comprises two monomers of IL-10 or two monomers of variant IL-10 that are fused together and one or more VH and VL regions. Each monomer is individually linked to one or more VH region and/or VL region of an antibody. When more than one VH and/or VL region is used in this fusion protein configuration, the VH and VL regions may be from the same antibody or from at least two different antibodies. In one particular configuration of this fusion protein, the VH or VL region is linked to the amino terminal end of a first monomer which is then linked by its carboxy end to the amino terminal end of a second monomer which is then linked to the amino terminal end of a VL or VH. Optionally, additional VH or VL regions may be linked to the amino or carboxy terminal ends, wherein the VH or VL regions may be from the same antibody or from a different antibody. Representative examples of the fusion protein described above may include the following configurations (see e.g., FIGS. 5 (d)-(f):

_{NH2}(Ab₁₋VH)_{COOH}-(linker)-_{NH2}(monoIL10)_{COOH}-(linker)-_{NH2}(monoIL10)_{COOH}-(linker)-_{NH2}(Ab₁₋VL)_{COOH}

The fusion protein described above will be capable of folding in a manner that allows the monomers of IL-10 to form a homodimer and the variable domains (VH and VL) of an antibody to form a functional ABS.

In another embodiment, the fusion protein comprises two monomers of IL-10 or two monomers of variant IL-10 located at the opposing terminal ends of the fusion protein and at least one VH and VL region, wherein the VH and VL regions are linked together. In this configuration, the VH and VL regions are fused together and each monomer is individually linked to either a VL region or a VH region of a first antibody. In this configuration, the IL-10 monomers or the monomers of variant IL-10 are each individually linked to either a VH or VL of a first antibody. A representative example of the fusion protein described above may include the following configurations (see, e.g., FIGS. 5(a)-(c)):

_{NH2}(monoIL10)_{COOH}-(linker)-_{NH2}(Ab₁₋VH)_{COOH}-(linker)-_{NH2}(Ab₁₋VL)_{COOH}-(linker)-_{NH2}(monoIL10)_{COOH} a)

_{NH2}(monoIL10)_{COOH}-(linker)-_{NH2}(Ab₁₋VL)_{COOH}-(linker)-_{NH2}(Ab₁₋VH)_{COOH}-(linker)-_{NH2}(monoIL10)_{COOH} b)

The monomer of IL-10 or the monomer of a variant IL-10 may be linked to the VH or VL sequence through a linker sequence. The linker can be a carboxy terminal linker linking a carboxy end of a variable chain region (VH or VL) to an amino terminal end of a monomer of IL-10 or a monomeric IL-10 variant molecule. Alternatively, the linker can be an amino terminal linker whereby the carboxy terminal end of a monomeric IL-10 or a monomeric IL-10 variant molecule is linked to the amino terminal end of a variable chain region (VH or VL).

Thus, in one form, the fusion protein comprises a monomeric IL-10 molecule or a variant IL-10 molecule linked to two variable regions from at least two different antibodies, wherein the two variable regions are configured as a VH region from a first antibody linked to a VL region from a second antibody or a VL from the first antibody linked to a VH from the second antibody. The fusion protein according to this form may include monomeric IL-10 molecule or a variant thereof including at least one amino acid substitution that increases or decreases affinity to an IL-10 receptor. The amino acid substitution impacting IL-10 receptor binding may occur in a human, CMV or EBV IL-10. The amino acid substitution may preferably be in an EBV IL-10 and include amino acid substitution at position 31, 75, or both. The amino acid substitutions may include any one or more of a V31 L or A75I substitution or both. In addition to the amino acid substitutions impacting IL-10 receptor binding affinity, the IL-10 variant may also include modifications that impact the inter-domain angle. In another embodiment, the fusion protein comprises a configuration selected from: (a) a VH region of the first antibody linked at its carboxy terminal end to an amino terminal end of a VL region of the second antibody subsequently linked to a carboxy terminal end of a monomer of IL-10 or a variant thereof; or (b) an IL-10 molecule or a variant thereof linked at its carboxy terminal end to an amino terminal end of a VH region of the second antibody subsequently linked to an amino terminal end of a VL region of the first antibody. These fusion protein configurations include, in one preferred embodiment a sequence of SEQ ID Nos.: 24-28, or 29. These fusion proteins sequences are capable of forming a complex wherein the monomers of IL-10 or monomers of variant IL-10 are capable of forming a homodimer. Such a complex may include and/or configured as a diabody complex.

In another form, the fusion protein may be fashioned as an immunoconjugate comprising a first fusion protein comprising at its amino terminal end a heavy chain variable region (VH) of a first antibody linked to a light chain variable region (VL) of a second antibody further linked to a monomer of IL-10; and a second fusion protein comprising at its amino-terminal end a monomer of IL-10 linked to a VH of the second antibody further linked to a VL of the first antibody, wherein the VH and VL of the first and second antibodies associate into a diabody and the monomers of IL-10 form a functional dimeric IL-10 molecule.

In another preferred embodiment, an immunoconjugate complex comprises a first fusion protein comprising at its amino terminal end a VH region of a first antibody and a monomeric IL-10 molecule linked by its carboxy terminal end; and a second fusion protein comprising at its amino terminal end a monomer of IL-10 linked to a VL of the first antibody, wherein the VH region of the first antibody associates with the VL region of first antibody thereby allowing the monomeric IL-10 molecules on each peptide chain to form a functional IL-10 dimer. The monomer(s) of IL-10 or monomer(s) variant IL-10 may include, as described above, amino acid modifications that impact IL-10 receptor binding and/or inter-domain angle.

In another preferred embodiment, an immunoconjugate complex comprises a first fusion protein comprising at its amino terminal end a VH region of a first antibody linked to a monomeric IL-10 molecule; and a second fusion protein comprising at its amino terminal end a monomer of IL-10 linked to a VL of the first antibody, wherein the VH region of the first antibody associates with the VL region of first antibody thereby allowing the monomeric IL-10 molecules on each peptide chain to form a functional IL-10 dimer.

In yet another embodiment, the immunoconjugate comprises at its amino terminal end a monomer of a first IL-10 (or IL-10 variant molecule) monomer linked to a VH region of a first antibody linked to a VL region of the first antibody linked to a monomer of a second IL-10 (or IL-10 variant molecule), wherein the two IL-10 monomers are able to associate together to form a functional dimer of IL-10. The VH and VL regions described are capable of forming an antigen binding site that specifically target an antigen (e.g., receptor, protein, nucleic acid, etc.). Thus, there are two chains, Chain 1 and Chain 2 which together for a fusion protein complex that creates a functioning IL-10 (or IL-10 variant molecule) homodimer. Representative fusion protein chains (i.e., Chain 1 and Chain 2) are described in Table 2:

TABLE 1

| Chain 1 | Chain 2 |
|---|---|
| SEQ ID No: 24 | SEQ ID No: 25 |
| SEQ ID No: 26 | SEQ ID No: 27 |
| SEQ ID No: 28 | SEQ ID No: 29 |
| SEQ ID No: 35 | SEQ ID No: 36 |
| SEQ ID No: 38 | SEQ ID No: 39 |
| SEQ ID No: 41 | SEQ ID No: 42 |
| SEQ ID No: 46 | SEQ ID No: 47 |
| SEQ ID No: 48 | SEQ ID No: 49 |
| SEQ ID No: 50 | SEQ ID No: 51 |

The fusion proteins include a VH and VL from at least one antibody. The fusion protein may comprises a range of 1-4 variable regions. The variable regions may be from the same antibody or from at least two different antibodies. The antibody variable chains can be obtained or derived from a plurality of antibodies (e.g., those targeting proteins, cellular receptors, and/or tumor associated antigens). In another embodiment, the variable regions are obtained from antibodies that target antigens associated with various diseases (e.g., cancer) or those that are not typically found or rarely found in the serum of a healthy subject, for example variable regions from antibodies directed to EGFR, MadCam, HIV and/or Ebola. Thus, in one embodiment, the variable regions are obtained or derived from anti-EGFR, anti-MadCam, anti-HIV (Chan et al, J. Virol, 2018, 92(18):e006411-19) or anti-Ebola (US Published Application 2018/0180614, incorporated by reference in its entirety, especially mAbs described in Tables 2, 3, and 4) antibodies, for example. In another embodiment, the variable regions are obtained or derived from antibodies capable of enriching the concentration of cytokines, such as IL-10, to a specific target area so as to enable IL-10 to elicit its biological effect. Such an antibody might include those that target overexpressed or upregulated receptors or antigens in certain diseased regions or those that are specifically expressed in certain impacted areas. For example, the variable regions might be obtained from antibodies specific for epidermal growth factor receptor (EGFR); CD52; various immune check point targets, such as but not limited to PD-L1, PD-1, TIM3, BTLA, LAG3 or CTLA4; CD20; CD47; GD-2; HER2; GPC3; EpCAM; FAPa; 5T4; Trop2; EDB-FN; TGFβ Trap; MadCam, β7 integrin subunit; α4β7 integrin; α4 integrin SR-A1; SR-A3; SR-A4; SR-A5; SR-A6; SR-B; dSR-C1; SR-D1; SR-E1; SR-F1; SR-F2; SR-G; SR-H1; SR-H2; SR-I1; and SR-J1 to name a few. A monomer of IL-10 (e.g., human, CMV, or EBV) or variant IL-10 molecule (described herein) is conjugated to either the amino terminal end or the carboxy terminal end of a variable region (VH or VL), such that the IL-10 or variant IL-10 molecule is able to dimerize with one another.

The fusion protein or fusion protein complex may also have an antigen targeting functionality. The fusion protein or fusion protein complex will comprise VH and VL regions that are able to associate together to form an antigen binding site or ABS. In some configurations, the IL-10 or IL-10 variant molecule or monomers thereof will be covalently linked to the end comprising the antigen binding site. These targeting fusion proteins may comprise at least one functioning variable region or paired VH and VL at one end of the fusion protein such that the fusion protein retains the capacity to target an antigen as well as having a functioning homodimer of an IL-10 or IL-10 variant molecule (see, FIGS. 4 (a)-(f) and 5 (a)-(f)). The variable regions may be further modified (e.g., by addition, subtraction, or substitution) by altering one or more amino acid that reduce antigenicity in a subject. The VH and VL pair form a scaffolding onto which CDR regions obtained for a plurality of antibodies can be grafted. Such antibody CDR regions include those antibodies known and described above. For example, the CDR regions from any antibody may be grafted onto a VH and VL pair such as those described in SEQ ID Nos: 37, 44, or 45 or those fusion proteins that are capable of forming a fusion protein complex such as those described in SEQ ID Nos: 46 and 47; 48 and 49; or 50 and 51. The CDR regions in the above described VH and VL scaffolding will include the following number of amino acid positions available for CDR engraftment/insertion (Table 3):

TABLE 2

| | |
|---|---|
| Heavy chain CDR1 | 3-7 amino acids |
| Heavy chain CDR2 | 7-11 amino acids |
| Heavy chain CDR3 | 7-11 amino acids |
| Light chain CDR1 | 9-14 amino acids |
| Light chain CDR2 | 5-9 amino acids |
| Light chain CDR3 | 7-11 amino acids |

In another aspect, the fusion protein described above may be represented by one of the following general formula:

1) IL10-L¹-X¹-L¹-X²-L₁-IL10 (Formula I);

2) (Z)ₙ-X¹-L²-Y²-L¹-IL10 (Formula II);

3) IL10-L¹-Y¹-L²-X²-(Z)ₙ (Formula III);

4) X¹-L²-X²-L¹-IL10 (Formula IV);

5) IL10-L¹-X¹-L²-X² (Formula V);

6) X¹-L¹-IL10 (Formula VI); and/or

7) IL10-L¹-X² (Formula VII)

wherein
“IL-10” is human Il-10 (SEQ ID No: 1); EBV IL-10 (SEQ ID No: 3), DV05 (SEQ ID No:14 or 18), DV06 (SEQ ID No: 15 or 19), or DV07 (SEQ ID No:16 or 20);
“L₁” is a linker of SEQ ID No: 31;
“L₂” is a linker of SEQ ID No: 30;
“X₁” is a VH region obtained from a first antibody specific for epidermal growth factor receptor (EGFR); CD52; various immune check point targets, such as but not limited to PD-L1, PD-1, TIM3, BTLA, LAG3 or CTLA4; CD20; CD47; GD-2; HER2; GPC3; EpCAM; ICAM (ICAM-1, -2, -3, -4, -5), VCAM, FAPα; 5T4; Trop2; EDB-FN; TGFβ Trap; MadCam, β7 integrin subunit; a4β7 integrin; α4 integrin SR-A1; SR-A3; SR-A4; SR-A5; SR-A6; SR-B; dSR-C1; SR-D1; SR-E1; SR-F1; SR-F2; SR-G; SR-H1; SR-H2; SR-I1; SR-J1; HIV, or Ebola;
“X₂” is a VL region obtained from the same antibody as X₁;
“Y₁” is VH region obtained from a second antibody specific for epidermal growth factor receptor (EGFR); CD52; various immune check point targets, such as but not limited to PD-L1, PD-1, TIM3, BTLA, LAG3 or CTLA4; CD20; CD47; GD-2; HER2; GPC3; EpCAM; ICAM (ICAM-1, -2, -3, -4, -5), VCAM, FAPa; 5T4; Trop2; EDB-FN; TGFβ Trap; MadCam, β7 integrin subunit; a4β7 integrin; a4 integrin SR-A1; SR-A3; SR-A4; SR-A5; SR-A6; SR-B; dSR-C1; SR-D1; SR-E1; SR-F1; SR-F2; SR-G; SR-H1; SR-H2; SR-I1; SR-J1; HIV, or Ebola;
“Y₂” is a VL region obtained from the same antibody as Y₁;
wherein X and Y are obtained from the same or different antibody;
“Z” is a cytokine selected from IL-6, IL-4, IL-1, IL-2, IL-3, IL-5, IL-7, IL-8, IL-9, IL_15, IL-26, IL-27, IL-28, IL-29, GM-CSF, G-CSF, interferons-α, -β, -γ, EPO, TGF-β, or tumor necrosis factors-α, -β, basic FGF, EGF, PDGF, IL-4, IL-11, or IL-13;
“n” is an integer selected from 0-2.

In an embodiment, the substituents of Formula I-VII, above, is preferably selected from the following: IL-10 is preferably DV05, DV06, or DV07; X₁ and X2 are preferably an anti-EGFR, anti-PDGFR, anti-FGFR, anti-VEGF, anti-Her2Neu, anti-GPC3, anti-MAdCAM, anti-ICAM-1, -2, -3, -4, anti-VCAM, anti-HIV, or anti-Ebola; Y1 and Y2 are preferably anti-EGFR, anti-MAdCAM, anti-ICAM-1, -2,-3, -4, anti-VCAM, anti-HIV, or anti-Ebola; Z is selected from IL-2, IL-7, or IL-15; and n is 1. In a most preferred embodiment, the fusion proteins are anyone of SEQ ID Nos: 33-51. Those of skill in the art will understand that the presence of a Histidine tag is used in the purification process of the fusion protein and maybe left intact or removed from the final product. Those of skill in the art will also understand that the VH and VL framework regions of any of the antibodies described above may be substituted with other complementary-determining regions (CDR) regions. For example, if the VH and VL regions are from an anti-Ebola antibody, the six CDR regions (i.e., CDRs 1-3 of both the VH and VL) may be substituted with the 6 CDR regions of an anti-EGFR antibody (e.g., cetuximab). Thus, in one preferred embodiment, the fusion protein is SEQ ID Nos: 34-36. In another preferred embodiment, the fusion protein is one having the scaffolding represented by SEQ ID Nos: 37; 44; 45; 46-47; 48-49; or 50-51, where any 6 CDR regions from any antibody may be grafted. In other preferred embodiments, the CDR regions from the VH and VL regions of an anti-Ebola antibody may be grafted with the CDR regions from an anti-MAdCAM, anti-VCAM, or anti-ICAM-1, -2, -3, -4 antibody, wherein in a preferred embodiment the CDR regions may be grafted into a fusion protein of SEQ ID No: 37. The fusion proteins as described in the formulas II and III; formulas IV and V; and formulas VI and VII, above are designed to associate together to form a biologically active homodimer of IL-10 (or variant thereof). The fusion proteins described above are designed to either be non-targeting or targeting depending on the pair of VH and VL regions selected and/or the CDR regions engrafted into the VH and VL. The term “non-targeting” is meant to describe a VH and VL region that is not able to target to a specific antigen located in vivo because the antigen is not present or the antigen binding site (ABS) has been disabled or modified to eliminate the ABS functionality.

The fusion proteins described above may also include additional amino acid sequences that aid in the recovery or purification of the fusion proteins during the manufacturing process. These may include various affinity tags, such as but not limited to protein A, albumin-binding protein, alkaline phosphatase, FLAG epitope, galactose-binding protein, histidine tags, and any other tags that are well known in the art. See, e.g., Kimple et al (Curr. Protoc. Protein Sci., 2013, 73:Unit 9.9, Table 9.91, incorporated by reference in its entirety). In one aspect, the affinity tag is an histidine tag having an amino acid sequence of HHHHHH (SEQ ID No.: 32). The histidine tag may be removed or left intact from the final product. In another embodiment, the affinity tag is a protein A modification that is incorporated into the fusion protein (e.g., into the VH region of the fusion proteins described herein), such as those described in SEQ ID Nos: 34 or 44-51. A person of skill in the art will understand that any fusion protein sequence described herein can be modified to incorporate a protein A modification by inserting amino acid point substitutions within the antibody framework regions as described in the art.

### EXAMPLES

### Example 1—Monocyte/Macrophage Assessment

To determine the genetic markers associated with positive treatment to IL-10 or IL-10 based agent treatment, an assessment and classification of the phenotypic expression of various reactions to IL-10 is required. PBMCs were isolated from several healthy donors and diseased patients. The monocytes/macrophages were isolated and tested for (1) induction or production of IL-10 and (2) ability to reduce TNF-α secretion in response to IL-10.

IL-10 Induction, Production, or Secretion

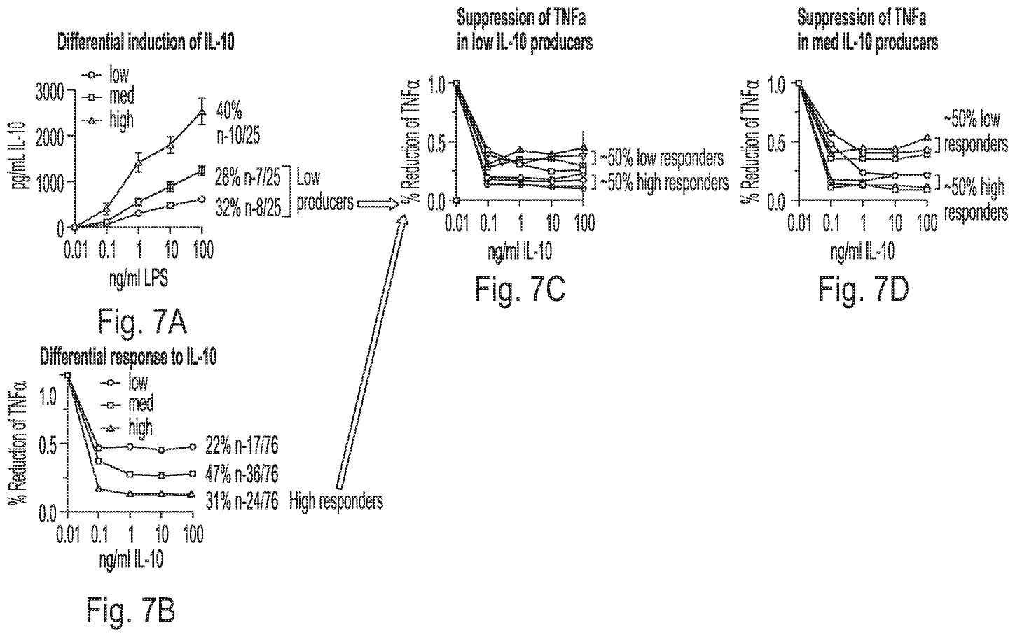

Peripheral blood monocytes/macrophages are isolated from whole peripheral blood by magnetic bead separation and plated at 2×10⁶ cells/mL in 200 μL in 96 well plates. Isolated peripheral blood monocytes/macrophages (2×10⁶) are also flash frozen and subjected to Next Generation Sequences (NGS) to confirm the gene expression and genetic signature associated with high, medium, or low response to and expression of IL-10. The isolated monocytes/macrophages were stimulated with a pro-inflammatory stimulant, LPS (0, 0.001, 0.01, 0.1, 1, 10, 100 ngs/mL IL-10 and 10 ngs/mL) for 18 hours. After 18 hours, supernatants are collected and the amount of IL-10 produced is measured. Referring to FIG. 7(a), LPS stimulated monocytes/macrophages from donors exhibited varying degrees IL-10 production, whereby approximately 40% produce high levels (greater than 2000 pg/ml) of IL-10, 28% produce medium levels (approximately 1000 pg/ml) of IL-10, and approximately 32% produced low levels of IL-10 (less than 1000 pg/ml).

### TNF-α Response

Likewise, the isolated monocytes/macrophages were also stimulated with LPS (10 ngs/ml) and IL-10 (0, 0.001, 0.01, 0.1, 1, 10, 100 ngs/mL) for 18 hours. After 18 hours, the supernatant is collected and assessed for IL-10 mediated control of inflammatory cytokine secretion. Referring to FIG. 7(b), IL-10 differentially reduced the levels of TNF-α in the donor pool. Approximately 22% were low responders (IL-10 capable of suppressing about 50% TNF-α secretion), 47% were medium responders (IL-10 capable of suppressing about 50-75% TNF-α secretion), and 31% were high responders (IL-10 capable of suppressing about greater than 75% TNF-α secretion).

Donor Stratification

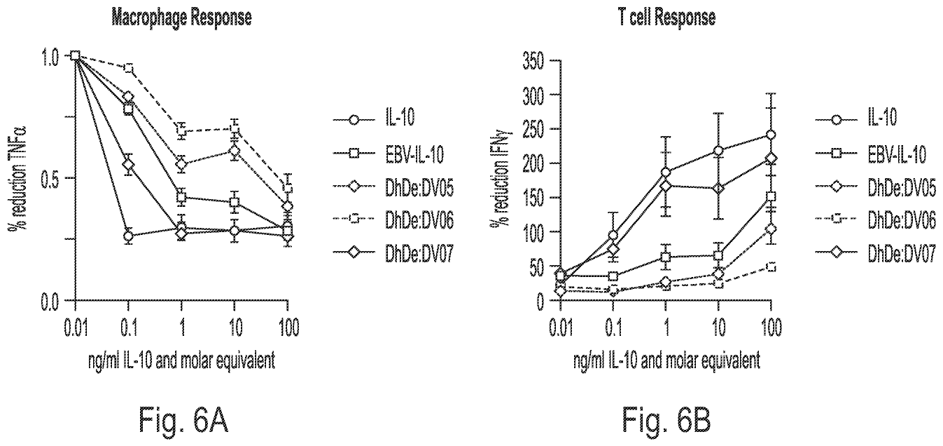

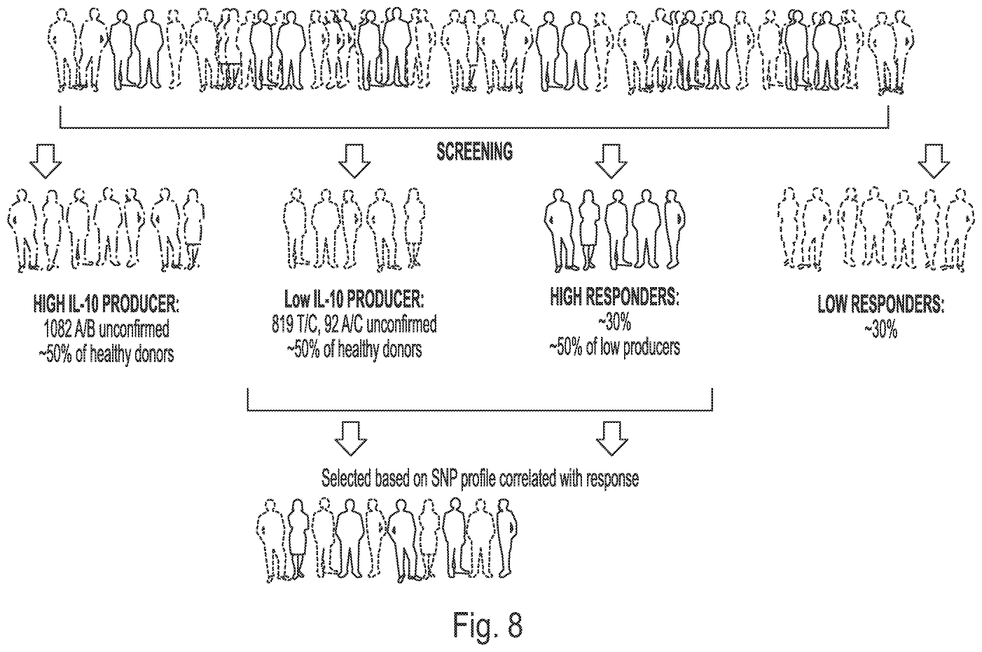

Those patients exhibiting both low production of IL-10 (i.e., low and medium IL-10 producer in FIG. 6(a)) and yet respond to IL-10 with the most significant reduction of TNF-α secretion (i.e. high responders in FIG. 6(b)) are the ideal candidates for IL-10 treatment. Approximately 50% of the donors are characterized as low/medium IL-10 producers and of the 50% low/medium IL-10 producers, half are high responders. (FIG. 7). Thus, the population to target would include those patients who have an ability to respond to IL-10 but are unable to endogenously produce high enough levels to treat inflammatory disease. Classification of donors exhibiting these two phenotypic traits will possess a genetic signature or make-up (e.g., gene expression profile and/or mutational frequency in form of SNP and/or INDEL signature) that is correlative to a group of patients that are positively receptive to treatment. (FIG. 8). The gene expression profiles and mutational frequency of those donors exhibiting these two phenotypic traits are then sequenced and the gene expression profile and/or SNP and/or INDEL profile from these donors are assessed to determine genetic patterns. It is those genetic patterns that will be the profile from which patients suffering from inflammatory disease will be most receptive and responsive to the administration of an IL-10 or IL-10 based agent therapy.

Crohn's Disease Assessment

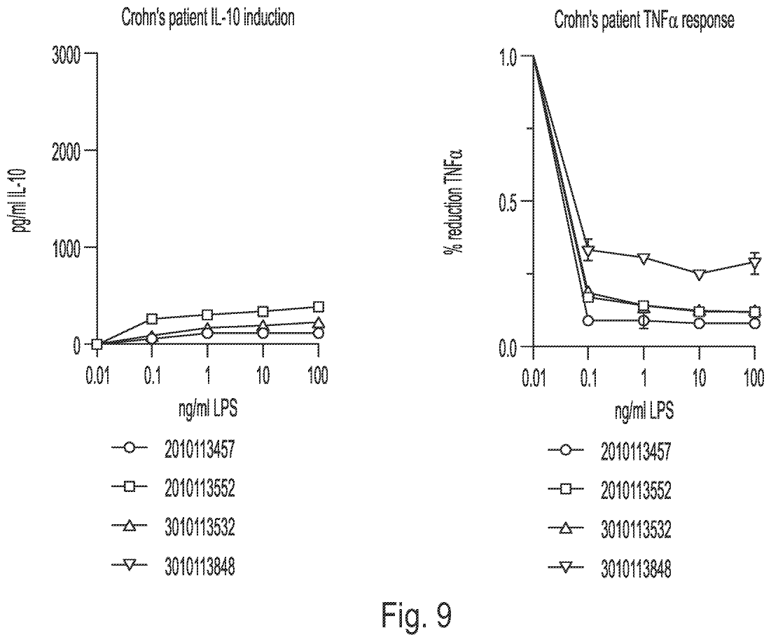

Monocytes/macrophages from Crohn's patients, were isolated and analyzed using (1) the IL-10 production assay and (2) TNF-α response assays. Referring to FIG. 9, all of the Crohn's patients are characterized as low producers of IL-10 (i.e., less than 1000 pg/ml) and 75% of Crohn's patients are characterized as high responders to IL-10 (wherein IL-10 is capable of suppressing about greater than 75% TNF-α secretion).

Accordingly, those patients exhibiting the combined phenotypic traits of low IL-10 induction, production, or secretion and high response to IL-10 (about 50% of the tested Crohn's patients) would have a genetic make-up similar to those screened from the healthy donor population. Thus, the genome of these Crohn's patients are sequenced and the genetic markers (gene expression signature and/or SNP and/or INDEL profile) from these donors are assessed to determine genetic patterns. These patterns are then cross-referenced with those from the healthy donor genetic profiles to result in a profile of potential target patients eligible and responsive to IL-10 reconstitutional therapy.

### Example 2

To determine whether patients can be classified/stratified based on their phenotypic response to IL-10 or IL-10 based agent, the monocytes/macrophage and T-cell response assays were used to assess both suppressive and stimulatory responses to IL-10. Multiple fresh donor peripheral blood samples were acquired to assess whether all human monocytes/macrophages and T cells respond to IL-10 in the same manner.

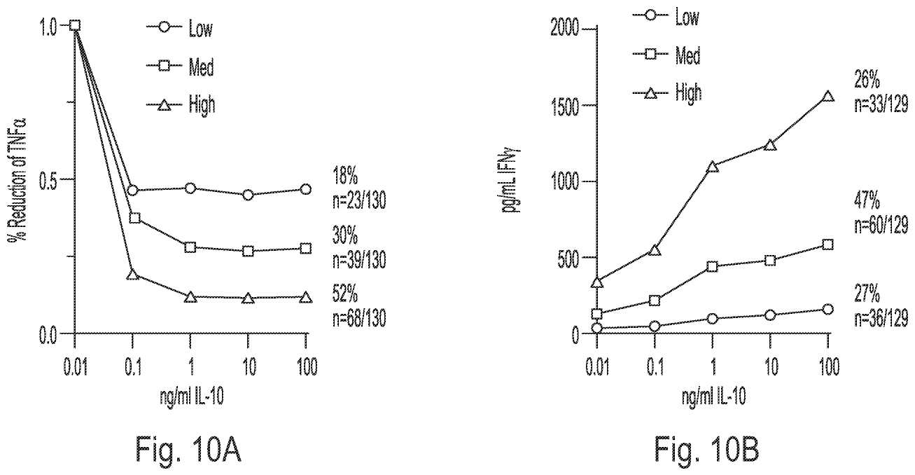

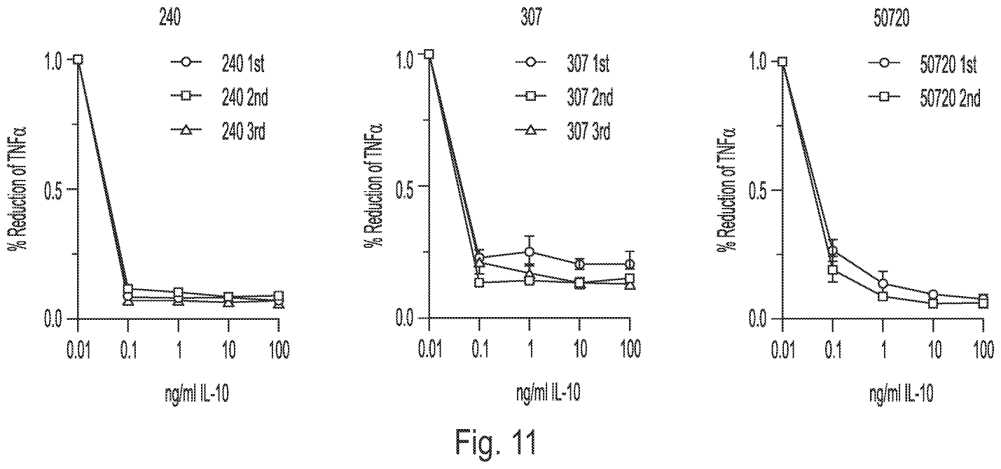

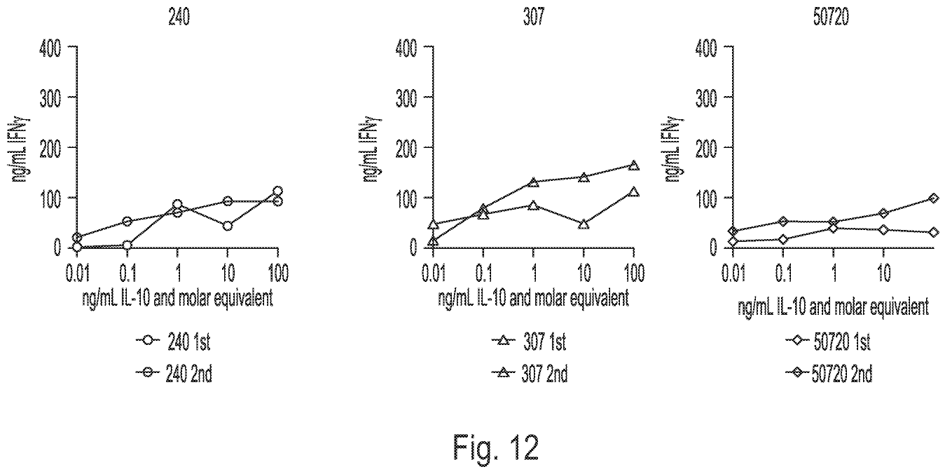

Data from 130 (monocyte) and 129 (CD8+ T cell) donor peripheral blood derived cellular responses to IL-10 suggest that there are differential magnitude of suppressive responses in monocytes to IL-10 and similarly differential magnitude of activating responses in CD8+ T cells to IL-10. See FIGS. 10A (monocyte) and 10B (T cells). To determine whether these responses were assay related, we procured repeated samples from the same donors. FIG. 11 shows the assessment of 3 donor monocyte responses to IL-10, while FIG. 12 show the assessment of 3 donor CD8+ T cell stimulatory responses to IL-10.

The data suggest that both monocytes/macrophages and CD8+ T cells responses to IL-10 are consistent across multiple testing time points. These data suggest it is possible that responses to IL-10 are controlled by combinations of donor or patient genetic factors that lead to differential expression profiles.

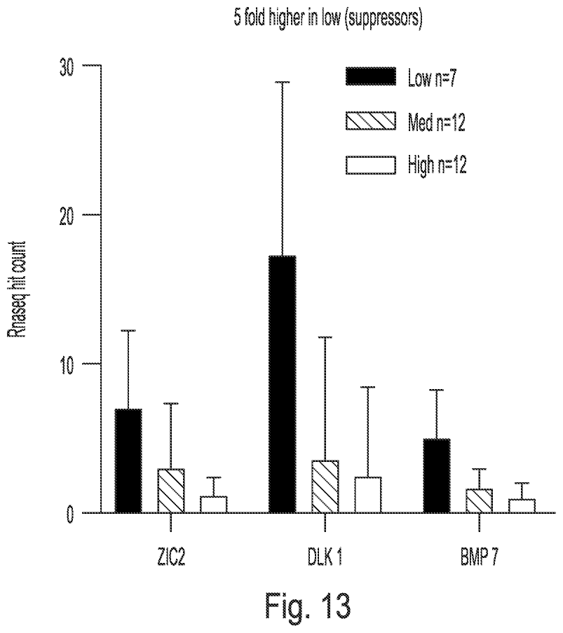

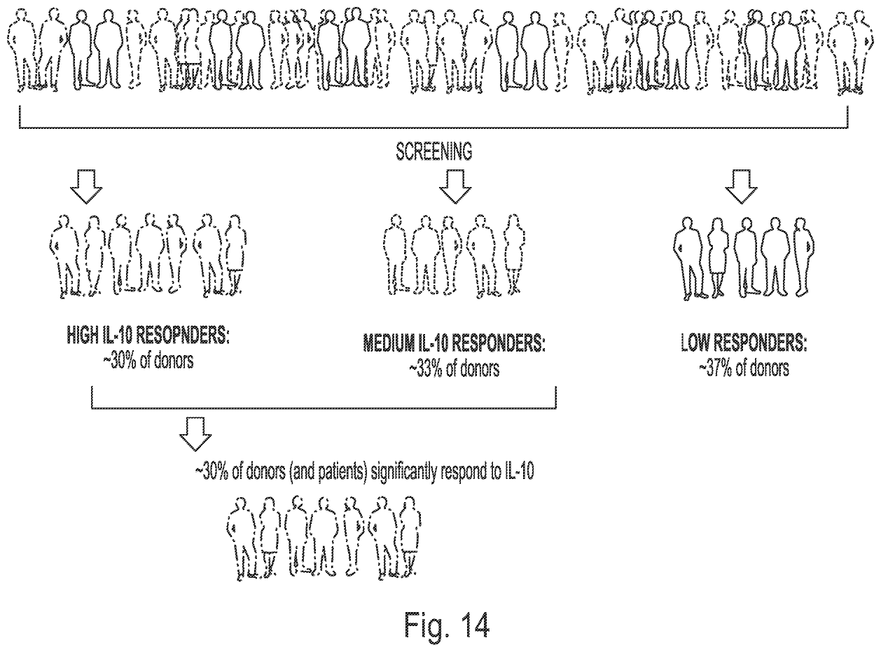

To evaluate this possibility, we utilized the CD8+ T cell assay to select donor CD8+ T cells that exhibit differential responses to IL-10 to perform next generation sequencing. We binned samples from high, medium and low responding donors to determine if there were any common genes expressed in any of the samples from the grouped responses. The results of the expression profiling analysis is provided in FIG. 13. The data illustrates that there are clear clusters of genes expressed in CD8+ T cells, for example from the non-responding donors. The same analysis was also performed on monocyctes/macrophages where samples were grouped from high, medium, and low secretion of IL-10 and high medium, and low response to IL-10 (resulting in a reduction of TNF-α secretion) in response to a pro-inflammatory stimulus, LPS.

We used the standard human reference genome to assess whether our samples had single nucleotide polymorphisms (SNP's) and/or RNA insertions or deletions (INDELs) via next generation sequencing. We then compiled the above gene expression analysis, SNP's and INDEL's and assessed whether the blended data clustered to known pathways using the Reactome Pathway database.

The blended data for T-cell high responders illustrated the following combined signatures (Table 3), where the genes identified by Hit Count (HC) (i.e., the mRNA expression level), INDEL (ID), and SNP (SNP) are provided:

TABLE 3
CD8+ T cells High Responder

| Molecular Pathway | Genes or expression profiles |
|---|---|
| gene and protein expression | HC.SERPINB 2, ID.PDCD4, SNP.HNRNPF, SNP.S OD2, |
| | SNP.STAT4 |
| chemokine receptors binding | HC.CCR1, HC .CXCL 13, HC.PPBP, SNP .CCL3L 1 |
| other semaphoring interactions | HC.TYROBP, ID.PTPRC, SNP.ITGB1, SNP.PTPRC |
| interleukin-10 signaling | HC.CCR1, HC.IL1B, SNP.CCL3L1, SNP.CSF2, SNP.TYK2. |
| | HC.CXCL10 |
| neutrophil degranulation | HC.HSPA6, HC.KRT1, HC.PPBP, HC.TYROBP, ID.ATP11A, |
| | ID.DYNLT1, ID.LPCAT1, ID. PAFAH1B 2, ID.PRKCD, |
| | ID.PTPRC, ID.RAP1A, SNP.CCT8, SNP.CD300A, |
| | SNP.CSNK2B, SNP.CYSTM1, SNP.DEGS1, SNP.ITGAM, |
| | SNP.NBEAL2, SNP.NBEAL2, SNP.PSMD3, SNP.PTPRC, |
| | SNP.ROCK1, SNP.VPS35L |
| interleukin-12 signaling | HC.SEPINB2, ID.PDCD4, SNP.HNRNPF, SNP.SOD2, |
| | SNP.STAT4, SNP.TYK2 |
| signaling by interleukins | HC.CCR1, HC.IL1B, HC.IL31, HC.IL5, HC.SERPINB 2, |
| | HC.CXCL10, HC.FOS, HC.IL9, ID.AKT1, ID.MEF2A, |
| | ID.PDCD4, SNP.ANXA1, SNP.CCL3L1, SNP.CSF2, |
| | SNP.HNRNPF, SNP.ITGAM, SNP.ITGB1, SNP.NFKB1A, |
| | SNP.P SMA1, SNP.PSMD3, SNP.SOD2, SNP.STAT4, |
| | SNP.STAT6, SNP.STX4, SNP.TYK2 SNP.ANXA1, ID.ANXA1 |
| interleukin-12 family signaling | HC.SERPINB2, ID.PDCD4, SNP.HNRNPF, SNP.SOD2, |
| | SNP.STAT4, SNP.TYK2 |
| Activation of ATR in response to | SNP.ORC3 |
| replication stress | |
| Activation of the AP-1 family of | HC.FOS |
| transcription factors | |
| Activation of the pre-replicative | SNP.ORC3 |
| complex | |
| Assembly of the ORC complex at | SNP.ORC3 |
| the origin of replication | |
| CDC6 association with the | SNP.ORC3 |
| ORC:origin complex | |
| Defective CYP19A1 causes | HC.CYP19A1 |
| Aromatase excess syndrome | |
| (AEXS) | |
| DNA Replication Pre-Initiation | SNP.ORC3 |
| E2F-enabled inhibition of pre- | SNP.ORC3 |
| replication complex formation | |
| Estrogen-dependent nuclear events | HC.FOS |
| downstream of ESR-membrane | |
| signaling | |
| Formyl peptide receptors bind | SNP.ANXA1, ID.ANXA1 |
| formyl peptides and many other | |
| ligands | |
| G2/M Checkpoints | SNP.ORC3 |
| Hormone ligand-binding receptors HC.GNRH2 | |
| Interleukin-4 and Interleukin-13 | SNP.ANXA1, ID.ANXA1, HC.FOS |
| signaling | |
| Nef mediated downregulation of | SNP.AP1S3 |
| MHC class I complex cell surface | |
| expression | |
| Tryptophan catabolism | HC.IDO1 |

The blended data for CD8+ T-cell low responders illustrated the following combined signatures (Table 4):

TABLE 4
CD8+ T cells Low Responders

| Molecular Pathway | Genes or expression profiles |
|---|---|
| elastic fiber formation | HC.BMP7, ID.ITGAV, SNP.MFAP3 |
| signaling by Notchl | HC.DLK1, ID.NCSTN, SNP.APH1B, |
| | SNP.ARB2, SNP.HDAC7, |
| | SNP.MAML2, SNP.MYC, SNP.PSEN2, |
| | SNP.UBA52 |
| activated Notchl transmission | HC.DLK1, ID.NCSTN, SNP.APH1B, |
| | SNP.ARRB2, |
| | SNP.PSEN2, SNP.UBA52 |
| CRMPs in Sema3A signaling | HC.DPYSL5 |
| POU5F1 (OCT4), | HC.SOX2 |
| SOX2, NANOG | |
| activate genes related to | |
| proliferation/differentiation | |
| Transcriptional regulation of | HC.LIN28A, HC.SOX2 |
| pluripotent stem cells | |

The blended data for Monocyte/Macrophage High IL-10 Production/Secretion illustrated the following combined signatures (Table 5):

TABLE 5
Monocyte/Macrophage cells High Producers/Secretors

| Molecular Pathway | Genes or expression profiles |
|---|---|
| Activation, translocation and | SNP.BAX, SNP.BID |
| oligomerization of BAX | |
| APC truncation mutants have | SNP.GSK3B, ID.GSK3B, SNP.PPP2CB, ID.PPP2CB, |
| impaired AXIN binding | SNP.PPP2R1A, SNP.PPP2R5C, ID.PPP2R5C |
| AXIN missense mutants destabilize | SNP.GSK3B, ID.GSK3B, SNP.PPP2CB, ID.PPP2CB, |
| the destruction complex | SNP.PPP2R1A, SNP.PPP2R5C, ID.PPP2R5C |
| GLI proteins bind promoters of Hh | HC.GLI1 , HC.PTCH2 |
| responsive genes to promote | |
| transcription | |
| LGI-ADAM interactions | HC.ADAM11, HC.ADAM23, HC.LGI4, HC.STX1A |
| Loss of MECP2 binding ability to | SNP.MECP2 |
| 5hmC-DNA | |
| Loss of MECP2 binding ability to | SNP.MECP2, SNP.NCOR1, ID.NCOR1 |
| the NCoR/SMRT complex | |
| MECP2 regulates transcription of | SNP.MECP2, HC.GAD1 |
| genes involved in GABA signaling | |
| Negative regulation of MAPK | SNP.KRAS, ID.KRAS, SNP.KSR1, SNP.PPP2CB, ID.PPP2CB, |
| pathway | SNP.PPP2R1A, SNP.PPP2R5C, ID.PPP2R5C, SNP.RAF1, |
| | ID.RAF1 |
| Regulation of MECP2 expression | SNP.AG03, ID.AG03, SNP.MECP2, SNP.NCOR1, ID.NCOR1, |
| and activity | SNP.TNRC6B, HC.CAMK2A |
| TICAM1, RIP1-mediated IKK | SNP.BIRC2, ID.BIRC2, SNP.TRAF6, SNP.UBE2D2 |
| complex recruitment | |
| truncated APC mutants destabilize | SNP.GSK3B, ID.GSK3B, SNP.PPP2CB, ID.PPP2CB, |
| the destruction complex | SNP.PPP2R1A, SNP.PPP2R5C, ID.PPP2R5C |

The blended data for Monocyte/Macrophage Low IL-10 Production/Secretion illustrated the following combined signatures (Table 6):

TABLE 6
Monocyte/Macrophage cells Low Secretors

| Molecular Pathway | Genes or expression profiles |
|---|---|
| Chemokine receptors bind | HC.CCL19, HC.CCR9, HC.CXCL11, HC.CXCL5, HC.CXCL6 |
| chemokines | |
| Hemostasis | SNP.FAM3C, HC.EGF, HC.EHD3, HC.ESAM, HC.F2RL3, |
| | HC.HBG1, HC.IGF2, HC.IGLV2-8, HC.ITGB3, HC.KIF28P, |
| | HC.MMRN1, HC.PF4V1, HC.PLA2G4A, HC.SELP, |
| | HC.SERPINE2, HC.SLC7A11, HC.SLC8A3, HC.THBS1 |
| Hydrolysis of LPC | HC.PLA2G4A |
| Insulin-like Growth Factor-2 | HC.IGF2 |
| mRNA Binding Proteins | |
| (IGF2BPs/IMPs/VICKZs) bind | |
| RNA | |
| Interleukin-35 Signaling | HC.EBI3 |
| Molecules associated with elastic | V.MFAP3, HC.ITGB3, HC.LTBP1 |
| fibers | |
| Non-integrin membrane-ECM | HC.FGF2, HC.ITGB3, HC.THBS1 |
| interactions | |
| Peptide ligand-binding receptors | HC.AVPR1A, HC.CCL19, HC.CCL23, HC.CCR9, HC.CXCL11, |
| | HC.CXCL5, HC.CXCL6, HC.F2RL3, HC.XK |
| Platelet degranulation | SNP.FAM3C, HC.EGF, HC.IGF2, HC.ITGB3, HC.MMRN1, |
| | HC.SELP, HC.THBS1 |
| Regulation of beta-cell | HC.HNF1B, HC.NR5A2 |
| development | |
| Regulation of gene expression in | HC.HNF1B, HC.NR5A2 |
| early pancreatic precursor cells | |
| Response to elevated platelet | SNP.FAM3C, HC.EGF, HC.IGF2, HC.ITGB3, HC.MMRN1, |
| cytosolic Ca2 | HC.SELP, HC.THBS1 |
| RUNX1 and FOXP3 control the | SNP.IL2RA, HC.IL2RA |
| development of regulatory T | |
| lymphocytes (Tregs) | |
| Syndecan interactions | HC.FGF2, HC.ITGB3, HC.THBS1 |

The blended data for Monocyte/Macrophage High IL-10 Response illustrated the following combined signatures (Table 7):

TABLE 6
Monocyte/Macrophage cells High Responders

| Molecular Pathway | Genes or expression profiles |
|---|---|
| Attenuation phase | SNP.DNAJB1, SNP.HSPA1L, SNP.HSPH1, HC.DNAJB1, |
| | HC.FKBP4, HC.HSPA1A, HC.HSPA1B, HC.HSPA1L, |
| | HC.HSPA6, HC.HSPH1, HC.SERPINH1 |
| Cellular response to heat stress | SNP.DNAJB1, SNP.HSPA1L, SNP.HSPH1, SNP.POM121, |
| | SNP.SEC13, HC.BAG3, HC.CAMK2A, HC.DNAJB1, |
| | HC.FKBP4, HC.HSPA1A, HC.HSPA1B, HC.HSPA1L, |
| | HC.HSPA6, HC.HSPA7, HC.HSPH1, HC.SERPINH1 |
| HSF1 activation | SNP.DNAJB1, SNP.HSPA1L, SNP.HSPH1, HC.DNAJB1, |
| | HC.FKBP4, HC.HSPA1A, HC.HSPA1B, HC.HSPA1L, |
| | HC.HSPA6, HC.HSPH1, HC.SERPINH1 |
| HSF1-dependent transactivation | SNP.DNAJB1, SNP.HSPA1L, SNP.HSPH1, HC.CAMK2A, |
| | HC.DNAJB1, HC.FKBP4, HC.HSPA1A, HC.HSPA1B, |
| | HC.HSPA1L, HC.HSPA6, HC.HSPH1, HC.SERPINH1 |
| Negative regulation of NMDA | SNP.LRRC7, SNP.PPM1F, HC.ACTN2, HC.CAMK1, |
| receptor-mediated neuronal | HC.CAMK2A, HC.DLG2, HC.GRIN2C, HC.GRIN2D |
| transmission | |
| Regulation of HSF1-mediated heat | SNP.DNAJB1, SNP.HSPA1L, SNP.HSPH1, SNP.POM121, |
| shock response | SNP.SEC13, HC.BAG3, HC.DNAJB1, HC.FKBP4, |
| | HC.HSPA1A, HC.HSPA1B, HC.HSPAlL, HC.HSPA6, |
| | HC.HSPA7, HC.HSPH1, HC.SERPINH1 |

The blended data for Monocyte/Macrophage Low IL-10 Response illustrated the following combined signatures (Table 8):

TABLE 8
Monocyte/Macrophage cells Low Responders

| Molecular Pathway | Genes or expression profiles |
|---|---|
| Classical antibody-mediated | HC.IGKV1D-16, HC.IGLV3-25, HC.IGLV4-69, HC.IGLV7-46 |
| complement activation | |
| Competing endogenous RNAs | SNP.TNRC6B, SNP.TNRC6C, ID.TNRC6C |
| (ceRNAs) regulate PTEN | |
| translation | |
| Creation of C4 and C2 activators | HC.IGKV1D-16, HC.IGLV3-25, HC.IGLV4-69, HC.IGLV7-46 |
| Defective LMBRD1 causes | SNP.LMBRD1 |
| methylmalonic aciduria and | |
| homocystinuria type cblF | |
| FCERI mediated Ca+2 | SNP.TXK, HC.IGKV1D-16, HC.IGLV3-25, HC.IGLV4-69, |
| mobilization | HC.IGLV7-46 |
| FCERI mediated MAPK activation | SNP.NRAS, HC.IGKV1D-16, HC.IGLV3-25, HC.IGLV4-69, |
| | HC.IGLV7-46 |
| FCGR activation | HC.IGKV1D-16, HC.IGLV3-25, HC.IGLV4-69, HC.IGLV7-46 |
| NR1H3 & NR1H2 regulate gene | SNP.TNRC6B, SNP.TNRC6C, ID.TNRC6C, HC.APOC1 |
| expression linked to cholesterol | |
| transport and effux | |
| Post-transcriptional silencing by | SNP.TNRC6B, SNP.TNRC6C, ID.TNRC6C |
| small RNAs | |
| Receptor-type tyrosine-protein | HC.PTPRD |
| phosphatases | |
| Regulation of MECP2 expression | SNP.HDAC2, SNP.TNRC6B, SNP.TNRC6C, ID.TNRC6C |
| and activity | |
| Regulation of RUNX1 Expression | SNP.CCND3, SNP.TNRC6B, SNP.TNRC6C, ID.TNRC6C |
| and Activity | |
| Role of LAT2/NTAL/LAB on | HC.IGKV1D-16, HC.IGLV3-25, HC.IGLV4-69, HC.IGLV7-46 |
| calcium mobilization | |
| Scavenging of heme from plasma | HC.IGKV1D-16, HC.IGLV3-25, HC.IGLV4-69, HC.IGLV7-46 |
| Signaling by BMP | SNP.BMPR2, HC.NOG |
| Transcriptional regulation by | SNP.CCND3, SNP.CDK7, SNP.DPY30, SNP.PBRM1, |
| RUNX1 | SNP.PHC3, SNP.TNRC6B, SNP.TNRC6C, ID.TNRC6C, |
| | HC.TP73 |
| Vitamin B2 (riboflavin) | HC. SLC52A1 |
| metabolism | |

The genome wide mutational frequencies were also assessed in CD8+ T cells and monocytes/macrophages that correlated to high and low responders to IL-10 in CD8+ T cells; high and low secretors of IL-10 in monocytes/macrophages following pro-inflammatory stimulus; and high and low responders to IL-10 following pro-inflammatory stimulus. The following tables 9-14 provide the gene, chromosome, chromosomal position, the reference sequence/nucleotide, and the mutations or alternative sequence (SNP or INDEL).

Lengthy table referenced here
US20210214782A1-20210715-T00001
Please refer to the end of the specification for access instructions.

Lengthy table referenced here
US20210214782A1-20210715-T00002
Please refer to the end of the specification for access instructions.

Lengthy table referenced here
US20210214782A1-20210715-T00003
Please refer to the end of the specification for access instructions.

Lengthy table referenced here
US20210214782A1-20210715-T00004
Please refer to the end of the specification for access instructions.

Lengthy table referenced here
US20210214782A1-20210715-T00005
Please refer to the end of the specification for access instructions.

Lengthy table referenced here
US20210214782A1-20210715-T00006
Please refer to the end of the specification for access instructions.

### REFERENCES

Chan, I. H. (2015). The Potentiation of IFNg and Induction of Cytotoxic Proteins by Pegylated IL-10 in Human CD8 T cells. Journal of Interferon and Cytokine Research.
Fedorak, R. N. (2000). Recombinant Human Interleukin 10 in the Treatment of Patients With Mild to Moderately Active Crohn's DIsease. Gastroenterology, 1473-1482.
Malefyt, R. d. (1993). Direct effects of IL-10 on subsets of human CD4 T cell clones and resting T cells—Specific inhibition of IL-2 production and proliferation. Journal of Immunology, 4754-4765.
Moore, K. W. (2001). Interleukin-10 and interleukin-10 receptor. Annu. Rev. Immunol. 683-765.
Mumm, J. B. (2011). IL-10 Elicits IFNg Dependent Tumor Immune Surveillance. Cancer Cell, 781-796.
Naing, A. (2016). Safety, Antitumor Activity, and Immune Activation of Pegylated Recombinant Human Interleukin-10 (AM0010) in Patients With Advanced Solid Tumors. Journal of Clinical Oncology.
Naing, A. (2018). PEGylated IL-10 (Pegilodecakin) Induces Systemic Immune Activation, CD8+ T Cell Invigoration and Polyclonal T Cell Expansion in Cancer Patients. Cancer Cell.
Schreiber, S. (2000). Safety and Efficacy of Recombinant Human Interleukin 10 in Chronic Active Crohn's Disease. Gastroenterology, 1461-1472.
Tilg, H. (2002). Treatment of Crohn's disease with recombinant human interleukin 10 induces the proinflammatory cytokine interferon gamma. Gut, 191-195.

LENGTHY TABLES
The patent application contains a lengthy table section. A copy of the table is available in electronic form from the USPTO web site (https://seqdata.uspto.gov/?pageRequest=docDetail&DocID=US20210214782A1). An electronic copy of the table will also be available from the USPTO upon request and payment of the fee set forth in 37 CFR 1.19(b)(3).

1. A method of genotyping a single nucleotide polymorphism (SNP) or insertion or deletion of bases (INDEL) to obtain a profile indicative of patient receptiveness to treatment with IL-10 or an IL-10 based agent comprising
contacting a cellular sample from a patient with a proinflammatory stimulus to elicit IL-10 induction;
measuring the levels of TNF-α production and the levels of IL-10 induction from the cellular sample;
selecting the patient sample exhibiting a dual phenotype of
high TNF-α reduction in response to IL-10 or an IL-10 based agent stimulation and
low IL-10 induction; and
sequencing the entire genome from the patient exhibiting the dual phenotype for the presence of a SNP and/or INDEL profile.

2. The method of claim 1, wherein the cellular sample is obtained from peripheral blood.

3. The method of claim 1, wherein the cellular sample is a macrophage.

4. The method of claim 1, wherein the IL-10 is an IL-10 derivative.

5. The method of claim 1, wherein the IL-10 agent is an IL-10 fusion protein, wherein the fusion protein is an IL-10 fused to a minibody or diabody.

6. The method of claim 1, wherein the proinflammatory stimulus is lipopolysaccharide (LPS).

7. The method of claim 1, wherein the treatment is for an inflammatory disease.

8. The method of claim 1, wherein the inflammatory disease is inflammatory bowel disease (IBD), Crohn's disease or ulcerative colitis.

9. The method of claim 1, wherein patient is healthy patient or diseased patient.

10. The method of claim 1, further comprising selecting a patient sample exhibiting low or medium CD8+ T cell IFN-γ secretion.

11. A method of genotyping a single nucleotide polymorphism (SNP) or insertion or deletion of bases (INDEL) to obtain a profile indicative of patient receptiveness to treatment with IL-10 or an IL-10 based agent comprising
contacting activated CD8+ T cells obtained from the patient with an amount of IL-10 or an IL-10 based agent to induce secretion of IFN-γ;
measuring the level of IFN-γ secretion;
selecting the patient sample exhibiting a phenotype of high and/or medium IFN-γ secretion by CD8+ T cells; and
sequencing the entire genome from the patient exhibiting the phenotype for the presence of a SNP and/or INDEL profile.

12. The method of claim 11, wherein the CD8+ T cells are purified from a peripheral blood sample.

13. The method of claim 12, wherein the CD8+ T cells are activated with an anti-CD3 antibody prior to stimulation by an IL-10 or IL-10 based agent.

14. The method of claim 12, wherein the IL-10 is an IL-10 derivative.

15. The method of claim 12, wherein the IL-10 agent is an IL-10 fusion protein, wherein the fusion protein is an IL-10 fused to a minibody or diabody.

16. The method of claim 11, wherein the treatment is for cancer or autoimmune diseases/disorders.

17. The method of claim 11, wherein patient is healthy patient or diseased patient.

18. The method of claim 11, wherein the profile is a gene and protein expression profile provided in Tables 3 and 4.

19. A method for treating a patient suffering from an disease or disorder alleviated by IL-10 or an IL-10 based agent comprising
genetically profiling a sample obtained from a diseased patient for a SNP/INDEL profile, wherein the SNP/INDEL profile is correlated with a phenotypic trait wherein the macrophages respond to IL-10 by reducing the level of TNF-α and produces low amounts of IL-10 in response to LPS stimulation;
selecting a patient possessing the SNP/INDEL profile; and
administering to the patient a therapeutically effective amount of an IL-10 or IL-10 based agent.

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)


SEQUENCE LISTING (69 sequences)

SEQ ID NO: 1 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 2 - DNA, 1629 bp, Homo sapiens
acacatcaggggcttgctcttgcaaaaccaaaccacaagacagacttgcaaaagaaggcatgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgagacatcagggtggcgactctatagactctaggacataaattagaggtctccaaaatcggatctggggctctgggatagctgacccagccccttgagaaaccttattgtacctctcttatagaatatttattacctctgatacctcaacccccatttctatttatttactgagcttctctgtgaacgatttagaaagaagcccaatattataatttttttcaatatttattattttcacctgtttttaagctgtttccatagggtgacacactatggtatttgagtgttttaagataaattataagttacataagggaggaaaaaaaatgttctttggggagccaacagaagcttccattccaagcctgaccacgctttctagctgttgagctgttttccctgacctccctctaatttatcttgtctctgggcttggggcttcctaactgctacaaatactcttaggaagagaaaccagggagcccctttgatgattaattcaccttccagtgtctcggagggattcccctaacctcattccccaaccacttcattcttgaaagctgtggccagcttgttatttataacaacctaaatttggttctaggccgggcgcggtggctcacgcctgtaatcccagcactttgggaggctgaggcgggtggatcacttgaggtcaggagttcctaaccagcctggtcaacatggtgaaaccccgtctctactaaaaatacaaaaattagccgggcatggtggcgcgcacctgtaatcccagctacttgggaggctgaggcaagagaattgcttgaacccaggagatggaagttgcagtgagctgatatcatgcccctgtactccagcctgggtgacagagcaagactctgtctcaaaaaataaaaataaaaataaatttggttctaatagaactcagttttaactagaatttattcaattcctctgggaatgttacattgtttgtctgtcttcatagcagattttaattttgaataaataaatgtatcttattcacatc

SEQ ID NO: 3 - protein, 147 aa, Human gammaherpesvirus 4
TDQCDNFPQMLRDLRDAFSRVKTFFQTKDEVDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 4 - DNA, 632 bp, Human gammaherpesvirus 4
tataaatcacttccctatctcaggtaggcctgcacaccttaggtatggagcgaaggttagtggtcactctgcagtgcctggtgctgctttacctggcacctgagtgtggaggtacagaccaatgtgacaattttccccaaatgttgagggacctaagagatgccttcagtcgtgttaaaacctttttccagacaaaggacgaggtagataaccttttgctcaaggagtctctgctagaggactttaagggctaccttggatgccaggccctgtcagaaatgatccaattctacctggaggaagtcatgccacaggctgaaaaccaggaccctgaagccaaagaccatgtcaattctttgggtgaaaatctaaagaccctacggctccgcctgcgcaggtgccacaggttcctgccgtgtgagaacaagagtaaagctgtggaacagataaaaaatgcctttaacaagctgcaggaaaaaggaatttacaaagccatgagtgaatttgacatttttattaactacatagaagcatacatgacaattaaagccaggtgataattccataccctggaagcaggagatgggtgcatttcaccccaaccccccctttcgactgtcatttacaataaa

SEQ ID NO: 5 - protein, 177 aa, Human betaherpesvirus 5
MLSVMVSSSLVLIVFFLGASEEAKPAATTTTIKNTKPQCRPEDYASRLQDLRVTFHRVKPTLQREDDYSVWLDGTVVKGCWGCSVMDWLLRRYLEIVFPAGDHVYPGLKTELHSMRSTLESIYKDMRQCPLLGCGDKSVISRLSQEAERKSDNGTRKGLSELDTLFSRLEEYLHSRK

SEQ ID NO: 6 - DNA, 693 bp, Human betaherpesvirus 5
atgctgtcggtgatggtctcttcctctctggtcctgatcgtcttttttctaggcgcttccgaggaggcgaagccggcggcgacgacgacgacgataaagaatacaaagccgcagtgtcgtccggaggattacgcgagcagattgcaagatctccgcgtcacctttcatcgagtaaaacctacgttggtaggtcatgtaggtacggtttattgcgacggtctttcttttccgcgtgtcgggtgacgtagttttcctcttgtagcaacgtgaggacgactactccgtgtggctcgacggtacggtggtcaaaggctgttggggatgcagcgtcatggactggttgttgaggcggtatctggagatcgtgttccccgcaggcgaccacgtctatcctggacttaagacggaattgcatagtatgcgctcgacgctagaatccatctacaaagacatgcggcaatgcgtaagtgtctctgtggcggcgctgtccgcgcagaggtaacaacgtgttcatagcacgctgttttacttttgtcgggctcccagcctctgttaggttgcggagataagtccgtgattagtcggctgtctcaggaggcggaaaggaaatcggataacggcacgcggaaaggtctcagcgagttggacacgttgtttagccgtctcgaagagtatctgcactcgagaaagtag

SEQ ID NO: 7 - protein, 178 aa, Mus musculus
MPGSALLCCLLLLTGMRISRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 8 - DNA, 1314 bp, Mus musculus
ggggggggggatttagagacttgctcttgcactaccaaagccacaaagcagccttgcagaaaagagagctccatcatgcctggctcagcactgctatgctgcctgctcttactgactggcatgaggatcagcaggggccagtacagccgggaagacaataactgcacccacttcccagtcggccagagccacatgctcctagagctgcggactgccttcagccaggtgaagactttctttcaaacaaaggaccagctggacaacatactgctaaccgactccttaatgcaggactttaagggttacttgggttgccaagccttatcggaaatgatccagttttacctggtagaagtgatgccccaggcagagaagcatggcccagaaatcaaggagcatttgaattccctgggtgagaagctgaagaccctcaggatgcggctgaggcgctgtcatcgatttctcccctgtgaaaataagagcaaggcagtggagcaggtgaagagtgattttaataagctccaagaccaaggtgtctacaaggccatgaatgaatttgacatcttcatcaactgcatagaagcatacatgatgatcaaaatgaaaagctaaaacacctgcagtgtgtattgagtctgctggactccaggacctagacagagctctctaaatctgatccagggatcttagctaacggaaacaactccttggaaaacctcgtttgtacctctctccgaaatatttattacctctgatacctcagttcccattctatttattcactgagcttctctgtgaactatttagaaagaagcccaatattataattttacagtatttattatttttaacctgtgtttaagctgtttccattggggacactttatagtatttaaagggagattatattatatgatgggaggggttcttccttgggaagcaattgaagcttctattctaaggctggccacacttgagagctgcagggccctttgctatggtgtcctttcaattgctctcatccctgagttcagagctcctaagagagttgtgaagaaactcatgggtcttgggaagagaaaccagggagatcctttgatgatcattcctgcagcagctcagagggttcccctactgtcatcccccagccgcttcatccctgaaaactgtggccagtttgttatttataaccacctaaaattagttctaatagaactcatttttaactagaagtaatgcaattcctctgggaatggtgtattgtttgtctgcctttgtagcagcatctaattttgaataaatggatcttattcg

SEQ ID NO: 9 - protein, 168 aa, Artificial Sequence
  Description of Artificial Sequence Synthetic polypeptide
MDMRVPAQLLGLLLLWLRGARCGTDQCDNFPQMLRDLRDAFSRVKTFFQTDEVDNLLLKESLLEDFKGYLGCQALSEMIQFYLEEVMPQAENQDPEAKDHVNSLGENLKLRLRLRRCHRFLPCENKSKAVEQIKNAFNKLQEKGIYKAMSEFDIFINYIEAYMTIKAR

SEQ ID NO: 10 - protein, 167 aa, Artificial Sequence
  Description of Artificial Sequence Synthetic polypeptide
MDMRVPAQLLGLLLLWLRGARCGTDQCDNFPQMLRDLRDAFSRVKTFFQTKDEVDNLLLKESLLEDFKGYLGCQALSEMIQFYLEEVMPQAENQDPEAKDHVNSLGENLKTLRLRLRQCPLLGCGDKAVEQIKNAFNKLQEKGIYKAMSEFDIFINYIEAYMTIKAR

SEQ ID NO: 11 - protein, 167 aa, Artificial Sequence
  Description of Artificial Sequence Synthetic polypeptide
MDMRVPAQLLGLLLLWLRGARCGTDQCDNFPQMLRDLRDAFSRVKTFFQTKDELDNLLLKESLLEDFKGYLGCQALSEMIQFYLEEVMPQAENQDPEAKDHVNSLGENLKTLRLRLRQCPLLGCGDKAVEQIKNAFNKLQEKGIYKAMSEFDIFINYIEAYMTIKAR

SEQ ID NO: 12 - protein, 167 aa, Artificial Sequence
  Description of Artificial Sequence Synthetic polypeptide
MDMRVPAQLLGLLLLWLRGARCGTDQCDNFPQMLRDLRDAFSRVKTFFQTKDEVDNLLLKESLLEDFKGYLGCQALSEMIQFYLEEVMPQAENQDPEIKDHVNSLGENLKTLRLRLRQCPLLGCGDKAVEQIKNAFNKLQEKGIYKAMSEFDIFINYIEAYMTIKAR

SEQ ID NO: 13 - protein, 167 aa, Artificial Sequence
  Description of Artificial Sequence Synthetic polypeptide
MDMRVPAQLLGLLLLWLRGARCGTDQCDNFPQMLRDLRDAFSRVKTFFQTKDELDNLLLKESLLEDFKGYLGCQALSEMIQFYLEEVMPQAENQDPEIKDHVNSLGENLKTLRLRLRQCPLLGCGDKAVEQIKNAFNKLQEKGIYKAMSEFDIFINYIEAYMTIKAR

SEQ ID NO: 14 - protein, 170 aa, Artificial Sequence
  Description of Artificial Sequence Synthetic polypeptide
MDMRVPAQLLGLLLLWLRGARCGTDQCDNFPQMLRDLRDAFSRVKTFFQTKDELDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 15 - protein, 170 aa, Artificial Sequence
  Description of Artificial Sequence Synthetic polypeptide
MDMRVPAQLLGLLLLWLRGARCGTDQCDNFPQMLRDLRDAFSRVKTFFQTKDEVDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKAR

SEQ ID NO: 16 - protein, 170 aa, Artificial Sequence
  Description of Artificial Sequence Synthetic polypeptide
MDMRVPAQLLGLLLLWLRGARCGTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKAR

SEQ ID NO: 17 - protein, 184 aa, Artificial Sequence
  Description of Artificial Sequence Synthetic polypeptide
MDMRVPAQLLGLLLLWLRGARCGTDQCDNFPQMLRDLRDAFSRVKTFFQTKDEVDNLLLKESLLEDFKGYLGCQALSEMIQFYLEEVMPQAENQDPEAKDHVNSLGENLKTLRLRLRRCHRFLPCENGGGSGGKSKAVEQIKNAFNKLQEKGIYKAMSEFDIFINYIEAYMTIKARDYKDDDDK

SEQ ID NO: 18 - protein, 184 aa, Artificial Sequence
  Description of Artificial Sequence Synthetic polypeptide
MDMRVPAQLLGLLLLWLRGARCGTDQCDNFPQMLRDLRDAFSRVKTFFQTKDELDNLLLKESLLEDFKGYLGCQALSEMIQFYLEEVMPQAENQDPEAKDHVNSLGENLKTLRLRLRRCHRFLPCENGGGSGGKSKAVEQIKNAFNKLQEKGIYKAMSEFDIFINYIEAYMTIKARDYKDDDDK

SEQ ID NO: 19 - protein, 184 aa, Artificial Sequence
  Description of Artificial Sequence Synthetic polypeptide
MDMRVPAQLLGLLLLWLRGARCGTDQCDNFPQMLRDLRDAFSRVKTFFQTKDEVDNLLLKESLLEDFKGYLGCQALSEMIQFYLEEVMPQAENQDPEIKDHVNSLGENLKTLRLRLRRCHRFLPCENGGGSGGKSKAVEQIKNAFNKLQEKGIYKAMSEFDIFINYIEAYMTIKARDYKDDDDK

SEQ ID NO: 20 - protein, 184 aa, Artificial Sequence
  Description of Artificial Sequence Synthetic polypeptide
MDMRVPAQLLGLLLLWLRGARCGTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENGGGSGGKSKAVEQIKNAFNKLQEKGIYKAMSEFDIFINYIEAYMTIKARDYKDDDDK

SEQ ID NO: 21 - protein, 192 aa, Artificial Sequence
  Description of Artificial Sequence Synthetic polypeptide
MDMRVPAQLLGLLLLWLRGARCGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRNDYKDDDDK

SEQ ID NO: 22 - protein, 186 aa, Artificial Sequence
  Description of Artificial Sequence Synthetic polypeptide
MDMRVPAQLLGLLLLWLRGARCGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNDYKDDDDK

SEQ ID NO: 23 - protein, 178 aa, Artificial Sequence
  Description of Artificial Sequence Synthetic polypeptide
MDMRVPAQLLGLLLLWLRGARCGTDQCDNFPQMLRDLRDAFSRVKTFFQTKDEVDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKARDYKDDDDK

SEQ ID NO: 24 - protein, 398 aa, Artificial Sequence
  Description of Artificial Sequence Synthetic polypeptide
QVQLVQSGAEVKKPGASVKISCKASGYNFTTYAMHWVRQAPGQGLEWMGWINGGNGDTRYSQKFRGRVTISRDTSASTAYMELHSLTSEDTALFYCARESGDYYSEISGALDWGQGTLVTVSSGGGGSEIVMTQSPGTLSLSPGERATLSCRASQSVPRNYIGWFQQKPGQAPRLLIYGASSRAAGFPDRFSGSGSGTDFTLTITRLEPEDFAMYYCHQYDRLPYTFGQGTKLEIKGGGGSGGGGSGGGGSTDQCDNFPQMLRDLRDAFSRVKTFFQTKDELDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 25 - protein, 397 aa, Artificial Sequence
  Description of Artificial Sequence Synthetic polypeptide
TDQCDNFPQMLRDLRDAFSRVKTFFQTKDELDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKARGGGGSGGGGSGGGGSQVQLQQWGAGLLKPSETLSLTCAVYGGSFTTTYWNWIRQPPGKGLEWIGEVNYSGNANYNPSLKGRVAISVDTSKNQFSLRLNSVTAADTAIYYCTSRIRSHIAYSWKGDVWGKGTTVTVSSGGGGSSYELTQPPSVSVSPGQTARITCSGDVLPKKYAYWYQQKSGLAPVLVIYEDNRRPSGIPERFSGSSSGTMATLTISGAQVEDEGDYYCSSTDSSGDHYVFGTGTKVTVL

SEQ ID NO: 26 - protein, 398 aa, Artificial Sequence
  Description of Artificial Sequence Synthetic polypeptide
QVQLVQSGAEVKKPGASVKISCKASGYNFTTYAMHWVRQAPGQGLEWMGWINGGNGDTRYSQKFRGRVTISRDTSASTAYMELHSLTSEDTALFYCARESGDYYSEISGALDWGQGTLVTVSSGGGGSEIVMTQSPGTLSLSPGERATLSCRASQSVPRNYIGWFQQKPGQAPRLLIYGASSRAAGFPDRFSGSGSGTDFTLTITRLEPEDFAMYYCHQYDRLPYTFGQGTKLEIKGGGGSGGGGSGGGGSTDQCDNFPQMLRDLRDAFSRVKTFFQTKDEVDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKAR

SEQ ID NO: 27 - protein, 397 aa, Artificial Sequence
  Description of Artificial Sequence Synthetic polypeptide
TDQCDNFPQMLRDLRDAFSRVKTFFQTKDEVDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSQVQLQQWGAGLLKPSETLSLTCAVYGGSFTTTYWNWIRQPPGKGLEWIGEVNYSGNANYNPSLKGRVAISVDTSKNQFSLRLNSVTAADTAIYYCTSRIRSHIAYSWKGDVWGKGTTVTVSSGGGGSSYELTQPPSVSVSPGQTARITCSGDVLPKKYAYWYQQKSGLAPVLVIYEDNRRPSGIPERFSGSSSGTMATLTISGAQVEDEGDYYCSSTDSSGDHYVFGTGTKVTVL

SEQ ID NO: 28 - protein, 398 aa, Artificial Sequence
  Description of Artificial Sequence Synthetic polypeptide
QVQLVQSGAEVKKPGASVKISCKASGYNFTTYAMHWVRQAPGQGLEWMGWINGGNGDTRYSQKFRGRVTISRDTSASTAYMELHSLTSEDTALFYCARESGDYYSEISGALDWGQGTLVTVSSGGGGSEIVMTQSPGTLSLSPGERATLSCRASQSVPRNYIGWFQQKPGQAPRLLIYGASSRAAGFPDRFSGSGSGTDFTLTITRLEPEDFAMYYCHQYDRLPYTFGQGTKLEIKGGGGSGGGGSGGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKAR

SEQ ID NO: 29 - protein, 397 aa, Artificial Sequence
  Description of Artificial Sequence Synthetic polypeptide
TDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSQVQLQQWGAGLLKPSETLSLTCAVYGGSFTTTYWNWIRQPPGKGLEWIGEVNYSGNANYNPSLKGRVAISVDTSKNQFSLRLNSVTAADTAIYYCTSRIRSHIAYSWKGDVWGKGTTVTVSSGGGGSSYELTQPPSVSVSPGQTARITCSGDVLPKKYAYWYQQKSGLAPVLVIYEDNRRPSGIPERFSGSSSGTMATLTISGAQVEDEGDYYCSSTDSSGDHYVFGTGTKVTVL

SEQ ID NO: 30 - protein, 7 aa, Artificial Sequence
  Description of Artificial Sequence Synthetic peptide
SSGGGGS

SEQ ID NO: 31 - protein, 15 aa, Artificial Sequence
  Description of Artificial Sequence Synthetic peptide
GGGGSGGGGSGGGGS

SEQ ID NO: 32 - protein, 6 aa, Artificial Sequence
  Description of Artificial Sequence Synthetic 6xHis tag
HHHHHH

SEQ ID NO: 33 - protein, 567 aa, Artificial Sequence
  Description of Artificial Sequence Synthetic polypeptide
TDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSQVQLQQWGAGLLKPSETLSLTCAVYGGSFTTTYWNWIRQPPGKGLEWIGEVNYSGNANYNPSLKGRVAISVDTSKNQFSLRLNSVTAADTAIYYCTSRIRSHIAYSWKGDVWGKGTTVTVGGGGSGGGGSGGGGSEIVMTQSPGTLSLSPGERATLSCRASQSVPRNYIGWFQQKPGQAPRLLIYGASSRAAGFPDRFSGSGSGTDFTLTITRLEPEDFAMYYCHQYDRLPYTFGQGTKLEIKGGGGSGGGGSGGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKAR

SEQ ID NO: 34 - protein, 561 aa, Artificial Sequence
  Description of Artificial Sequence Synthetic polypeptide
TDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSQVQLQQWGAGLLKPSETLRLTCAVYGGSFTNYGVHWIRQPPGKGLEWIGVIWSGGNTDYNTPFTSRVTISVDTSKNQFSLQLNSVTAADTAIYYCTSLTYYDYEFAWGKGTTVTVGGGGSGGGGSGGGGSEIVMTQSPGTLSLSPGERATLSCRASQSIGTNIHWFQQKPGQAPRLLIYYASESISGFPDRFSGSGSGTDFTLTITRLEPEDFAMYYCQQNNNWPTTFGQGTKLEIKGGGGSGGGGSGGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKAR

SEQ ID NO: 35 - protein, 396 aa, Artificial Sequence
  Description of Artificial Sequence Synthetic polypeptide
QVQLQQWGAGLLKPSETLSLTCAVYGGSFTNYGVHWIRQPPGKGLEWIGVIWSGGNTDYNTPFTSRVAISVDTSKNQFSLRLNSVTAADTAIYYCTSLTYYDYEFAWKGDVWGKGTTVTVSSGGGGSEIVMTQSPGTLSLSPGERATLSCRASQSIGTNIHWFQQKPGQAPRLLIYYASESISGFPDRFSGSGSGTDFTLTITRLEPEDFAMYYCQQNNNWPTTFGQGTKLEIKGGGGSGGGGSGGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKAR

SEQ ID NO: 36 - protein, 403 aa, Artificial Sequence
  Description of Artificial Sequence Synthetic polypeptide
TDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSQVQLQQWGAGLLKPSETLSLTCAVYGGSFNYGVHWIRQPPGKGLEWIGVIWSGGNTDYNTPFTSRVAISVDTSKNQFSLRLNSVTAADTAIYYCTSLTYYDYEFAWKGDVWGKGTTVTVSSGGGGSEIVMTQSPGTLSLSPGERATLSCRASQSIGTNIHWFQQKPGQAPRLLIYYASESISGFPDRFSGSGSGTDFTLTITRLEPEDFAMYYCQQNNNWPTTFGQGTKLEIKDYKDDDDK

SEQ ID NO: 37 - protein, 581 aa, Artificial Sequence
  Description of Artificial Sequence Synthetic polypeptide
  MOD_RES (193)..(199): Any amino acid
  MISC_FEATURE (193)..(199): This region may encompass 3-7 residues
  MOD_RES (214)..(231): Any amino acid
  MISC_FEATURE (214)..(231): This region may encompass 14-18 residues
  MOD_RES (264)..(274): Any amino acid
  MISC_FEATURE (264)..(274): This region may encompass 7-11 residues
  MOD_RES (329)..(342): Any amino acid
  MISC_FEATURE (329)..(342): This region may encompass 9-14 residues
  MOD_RES (358)..(366): Any amino acid
  MISC_FEATURE (358)..(366): This region may encompass 5-9 residues
  MOD_RES (399)..(409): Any amino acid
  MISC_FEATURE (399)..(409): This region may encompass 7-11 residues
  See specification as filed for detailed description of substitutions and preferred embodiments
TDQCDNFPQMLRDLRDAFSRVKTFFQTKDEVDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSQVQLQQWGAGLLKPSETLSLTCAVYGGSFTXXXXXXXWIRQPPGKGLEWIGXXXXXXXXXXXXXXXXXXRVAISVDTSKNQFSLRLNSVTAADTAIYYCTSXXXXXXXXXXXWKGDVWGKGTTVTVSSGGGGSGGGGSGGGGSEIVMTQSPGTLSLSPGERATLSCXXXXXXXXXXXXXXWFQQKPGQAPRLLIYXXXXXXXXXGFPDRFSGSGSGTDFTLTITRLEPEDFAMYYCXXXXXXXXXXXFGQGTKLEIKGGGGSGGGGSGGGGSTDQCDNFPQMLRDLRDAFSRVKTFFQTKDEVDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKAR

SEQ ID NO: 38 - protein, 397 aa, Artificial Sequence
  Description of Artificial Sequence Synthetic polypeptide
QVQLQQWGAGLLKPSETLSLTCAVYGGSFTTTYWNWIRQPPGKGLEWIGEVNYSGNANYNPSLKGRVAISVDTSKNQFSLRLNSVTAADTAIYYCTSRIRSHIAYSWKGDVWGKGTTVTVSSGGGGSEIVMTQSPGTLSLSPGERATLSCRASQSVPRNYIGWFQQKPGQAPRLLIYGASSRAAGFPDRFSGSGSGTDFTLTITRLEPEDFAMYYCHQYDRLPYTFGQGTKLEIKGGGGSGGGGSGGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKAR

SEQ ID NO: 39 - protein, 405 aa, Artificial Sequence
  Description of Artificial Sequence Synthetic polypeptide
TDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSQVQLQQWGAGLLKPSETLSLTCAVYGGSFTTTYWNWIRQPPGKGLEWIGEVNYSGNANYNPSLKGRVAISVDTSKNQFSLRLNSVTAADTAIYYCTSRIRSHIAYSWKGDVWGKGTTVTVSSGGGGSEIVMTQSPGTLSLSPGERATLSCRASQSVPRNYIGWFQQKPGQAPRLLIYGASSRAAGFPDRFSGSGSGTDFTLTITRLEPEDFAMYYCHQYDRLPYTFGQGTKLEIKDYKDDDDK

SEQ ID NO: 40 - protein, 569 aa, Artificial Sequence
  Description of Artificial Sequence Synthetic polypeptide
TDQCDNFPQMLRDLRDAFSRVKTFFQTKDEVDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSQVQLQQWGAGLLKPSETLSLTCAVYGGSFTTTYWNWIRQPPGKGLEWIGEVNYSGNANYNPSLKGRVAISVDTSKNQFSLRLNSVTAADTAIYYCTSRIRSHIAYSWKGDVWGKGTTVTVSSGGGGSGGGGSGGGGSEIVMTQSPGTLSLSPGERATLSCRASQSVPRNYIGWFQQKPGQAPRLLIYGASSRAAGFPDRFSGSGSGTDFTLTITRLEPEDFAMYYCHQYDRLPYTFGQGTKLEIKGGGGSGGGGSGGGGSTDQCDNFPQMLRDLRDAFSRVKTFFQTKDEVDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKAR

SEQ ID NO: 41 - protein, 399 aa, Artificial Sequence
  Description of Artificial Sequence Synthetic polypeptide
QVQLVQSGAEVKKPGASVKVSCKASGYTFTSYGINWVRQAPGQGLEWMGWISVYSGNTNYAQKVQGRVTMTADTSTSTAYMDLRSLRSDDTAVYYCAREGSSSSGDYYYGMDVWGQGTTVTVSSGGGGSEIVMTQSPGTLSLSPGERATLSCRASQSVPRNYIGWFQQKPGQAPRLLIYGASSRAAGFPDRFSGSGSGTDFTLTITRLEPEDFAMYYCHQYDRLPYTFGQGTKLEIKGGGGSGGGGSGGGGSTDQCDNFPQMLRDLRDAFSRVKTFFQTKDEVDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKAR

SEQ ID NO: 42 - protein, 401 aa, Artificial Sequence
  Description of Artificial Sequence Synthetic polypeptide
TDQCDNFPQMLRDLRDAFSRVKTFFQTKDEVDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSQVQLQQWGAGLLKPSETLSLTCAVYGGSFTTTYWNWIRQPPGKGLEWIGEVNYSGNANYNPSLKGRVAISVDTSKNQFSLRLNSVTAADTAIYYCTSRIRSHIAYSWKGDVWGKGTTVTVSSGGGGSDIVMTQTPLSLSVTPGQPASISCKSSQSLLHTDGTTYLYWYLQKPGQPPQLLIYEVSNRFSGVPDRFSGSGSGTDFTLKISRVEAEDVGIYYCMQNIQLPWTFGQGTKVEIK

SEQ ID NO: 43 - protein, 575 aa, Artificial Sequence
  Description of Artificial Sequence Synthetic polypeptide
TDQCDNFPQMLRDLRDAFSRVKTFFQTKDEVDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSQVQLVQSGAEVKKPGASVKVSCKASGYTFTSYGINWVRQAPGQGLEWMGWISVYSGNTNYAQKVQGRVTMTADTSTSTAYMDLRSLRSDDTAVYYCAREGSSSSGDYYYGMDVWGQGTTVTVSSGGGGSGGGGSGGGGSDIVMTQTPLSLSVTPGQPASISCKSSQSLLHTDGTTYLYWYLQKPGQPPQLLIYEVSNRFSGVPDRFSGSGSGTDFTLKISRVEAEDVGIYYCMQNIQLPWTFGQGTKVEIKGGGGSGGGGSGGGGSTDQCDNFPQMLRDLRDAFSRVKTFFQTKDEVDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKAR

SEQ ID NO: 44 - protein, 573 aa, Artificial Sequence
  Description of Artificial Sequence Synthetic polypeptide
  MOD_RES (193)..(199): Any amino acid
  MISC_FEATURE (193)..(199): This region may encompass 3-7 residues
  MOD_RES (213)..(230): Any amino acid
  MISC_FEATURE (213)..(230): This region may encompass 14-18 residues
  MOD_RES (263)..(273): Any amino acid
  MISC_FEATURE (263)..(273): This region may encompass 7-11 residues
  MOD_RES (321)..(334): Any amino acid
  MISC_FEATURE (321)..(334): This region may encompass 9-14 residues
  MOD_RES (350)..(358): Any amino acid
  MISC_FEATURE (350)..(358): This region may encompass 5-9 residues
  MOD_RES (391)..(401): Any amino acid
  MISC_FEATURE (391)..(401): This region may encompass 7-11 residues
  See specification as filed for detailed description of substitutions and preferred embodiments
TDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSQVQLQQWGAGLLKPSETLRLTCAVYGGSFTXXXXXXXWIRQPPGKGLEWIXXXXXXXXXXXXXXXXXXRVTISVDTSKNQFSLQLNSVTAADTAIYYCTSXXXXXXXXXXXWGKGTTVTVGGGGSGGGGSGGGGSEIVMTQSPGTLSLSPGERATLSCXXXXXXXXXXXXXXWFQQKPGQAPRLLIYXXXXXXXXXGFPDRFSGSGSGTDFTLTITRLEPEDFAMYYCXXXXXXXXXXXFGQGTKLEIKGGGGSGGGGSGGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKAR

SEQ ID NO: 45 - protein, 573 aa, Artificial Sequence
  Description of Artificial Sequence Synthetic polypeptide
  MOD_RES (193)..(199): Any amino acid
  MISC_FEATURE (193)..(199): This region may encompass 3-7 residues
  MOD_RES (213)..(230): Any amino acid
  MISC_FEATURE (213)..(230): This region may encompass 14-18 residues
  MOD_RES (263)..(273): Any amino acid
  MISC_FEATURE (263)..(273): This region may encompass 7-11 residues
  MOD_RES (321)..(334): Any amino acid
  MISC_FEATURE (321)..(334): This region may encompass 9-14 residues
  MOD_RES (350)..(358): Any amino acid
  MISC_FEATURE (350)..(358): This region may encompass 5-9 residues
  MOD_RES (391)..(401): Any amino acid
  MISC_FEATURE (391)..(401): This region may encompass 7-11 residues
  See specification as filed for detailed description of substitutions and preferred embodiments
TDQCDNFPQMLRDLRDAFSRVKTFFQTKDELDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKARGGGGSGGGGSGGGGSQVQLQQWGAGLLKPSETLRLTCAVYGGSFTXXXXXXXWIRQPPGKGLEWIXXXXXXXXXXXXXXXXXXRVTISVDTSKNQFSLQLNSVTAADTAIYYCTSXXXXXXXXXXXWGKGTTVTVGGGGSGGGGSGGGGSEIVMTQSPGTLSLSPGERATLSCXXXXXXXXXXXXXXWFQQKPGQAPRLLIYXXXXXXXXXGFPDRFSGSGSGTDFTLTITRLEPEDFAMYYCXXXXXXXXXXXFGQGTKLEIKGGGGSGGGGSGGGGSTDQCDNFPQMLRDLRDAFSRVKTFFQTKDELDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 46 - protein, 402 aa, Artificial Sequence
  Description of Artificial Sequence Synthetic polypeptide
  MOD_RES (31)..(37): Any amino acid
  MISC_FEATURE (31)..(37): This region may encompass 3-7 residues
  MOD_RES (52)..(62): Any amino acid
  MISC_FEATURE (52)..(62): This region may encompass 7-11 residues
  MOD_RES (95)..(105): Any amino acid
  MISC_FEATURE (95)..(105): This region may encompass 7-11 residues
  MOD_RES (150)..(163): Any amino acid
  MISC_FEATURE (150)..(163): This region may encompass 9-14 residues
  MOD_RES (179)..(187): Any amino acid
  MISC_FEATURE (179)..(187): This region may encompass 5-9 residues
  MOD_RES (220)..(230): Any amino acid
  MISC_FEATURE (220)..(230): This region may encompass 7-11 residues
  See specification as filed for detailed description of substitutions and preferred embodiments
QVQLQQWGAGLLKPSETLRLTCAVYGGSFTXXXXXXXWIRQPPGKGLEWIGXXXXXXXXXXXRVTISVDTSKNQFSLQLNSVTAADTAIYYCTSXXXXXXXXXXXWKGDVWGKGTTVTVSSGGGGSEIVMTQSPGTLSLSPGERATLSCXXXXXXXXXXXXXXWFQQKPGQAPRLLIYXXXXXXXXXGFPDRFSGSGSGTDFTLTITRLEPEDFAMYYCXXXXXXXXXXXFGQGTKLEIKGGGGSGGGGSGGGGSTDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKAR

SEQ ID NO: 47 - protein, 409 aa, Artificial Sequence
  Description of Artificial Sequence Synthetic polypeptide
  MOD_RES (192)..(198): Any amino acid
  MISC_FEATURE (192)..(198): This region may encompass 3-7 residues
  MOD_RES (213)..(223): Any amino acid
  MISC_FEATURE (213)..(223): This region may encompass 7-11 residues
  MOD_RES (256)..(266): Any amino acid
  MISC_FEATURE (256)..(266): This region may encompass 7-11 residues
  MOD_RES (311)..(324): Any amino acid
  MISC_FEATURE (311)..(324): This region may encompass 9-14 residues
  MOD_RES (340)..(348): Any amino acid
  MISC_FEATURE (340)..(348): This region may encompass 5-9 residues
  MOD_RES (381)..(391): Any amino acid
  MISC_FEATURE (381)..(391): This region may encompass 7-11 residues
  See specification as filed for detailed description of substitutions and preferred embodiments
TDQCDNFPQMLRDLRDAFSRVKTFFQTKDELDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSQVQLQQWGAGLLKPSETLRLTCAVYGGSFXXXXXXXWIRQPPGKGLEWIGXXXXXXXXXXXRVTISVDTSKNQFSLQLNSVTAADTAIYYCTSXXXXXXXXXXXWKGDVWGKGTTVTVSSGGGGSEIVMTQSPGTLSLSPGERATLSCXXXXXXXXXXXXXXWFQQKPGQAPRLLIYXXXXXXXXXGFPDRFSGSGSGTDFTLTITRLEPEDFAMYYCXXXXXXXXXXXFGQGTKLEIKDYKDDDDK

SEQ ID NO: 48 - protein, 402 aa, Artificial Sequence
  Description of Artificial Sequence Synthetic polypeptide
  MOD_RES (31)..(37): Any amino acid
  MISC_FEATURE (31)..(37): This region may encompass 3-7 residues
  MOD_RES (52)..(62): Any amino acid
  MISC_FEATURE (52)..(62): This region may encompass 7-11 residues
  MOD_RES (95)..(105): Any amino acid
  MISC_FEATURE (95)..(105): This region may encompass 7-11 residues
  MOD_RES (150)..(163): Any amino acid
  MISC_FEATURE (150)..(163): This region may encompass 9-14 residues
  MOD_RES (179)..(187): Any amino acid
  MISC_FEATURE (179)..(187): This region may encompass 5-9 residues
  MOD_RES (220)..(230): Any amino acid
  MISC_FEATURE (220)..(230): This region may encompass 7-11 residues
  See specification as filed for detailed description of substitutions and preferred embodiments
QVQLQQWGAGLLKPSETLRLTCAVYGGSFTXXXXXXXWIRQPPGKGLEWIGXXXXXXXXXXXRVTISVDTSKNQFSLQLNSVTAADTAIYYCTSXXXXXXXXXXXWKGDVWGKGTTVTVSSGGGGSEIVMTQSPGTLSLSPGERATLSCXXXXXXXXXXXXXXWFQQKPGQAPRLLIYXXXXXXXXXGFPDRFSGSGSGTDFTLTITRLEPEDFAMYYCXXXXXXXXXXXFGQGTKLEIKGGGGSGGGGSGGGGSTDQCDNFPQMLRDLRDAFSRVKTFFQTKDEVDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKAR

SEQ ID NO: 49 - protein, 409 aa, Artificial Sequence
  Description of Artificial Sequence Synthetic polypeptide
  MOD_RES (192)..(198): Any amino acid
  MISC_FEATURE (192)..(198): This region may encompass 3-7 residues
  MOD_RES (213)..(223): Any amino acid
  MISC_FEATURE (213)..(223): This region may encompass 7-11 residues
  MOD_RES (256)..(266): Any amino acid
  MISC_FEATURE (256)..(266): This region may encompass 7-11 residues
  MOD_RES (311)..(324): Any amino acid
  MISC_FEATURE (311)..(324): This region may encompass 9-14 residues
  MOD_RES (340)..(348): Any amino acid
  MISC_FEATURE (340)..(348): This region may encompass 5-9 residues
  MOD_RES (381)..(391): Any amino acid
  MISC_FEATURE (381)..(391): This region may encompass 7-11 residues
  See specification as filed for detailed description of substitutions and preferred embodiments
TDQCDNFPQMLRDLRDAFSRVKTFFQTKDEVDNLLLKESLLEDFKGYLGCQALSEMIQFYLEEVMPQAENQDPEIKDHVNSLGENLKTLRLRLRRCHRFLPCENKSKAVEQIKNAFNKLQEKGIYKAMSEFDIFINYIEAYMTIKARGGGGSGGGGSGGGGSQVQLQQWGAGLLKPSETLRLTCAVYGGSFXXXXXXXWIRQPPGKGLEWIGXXXXXXXXXXXRVTISVDTSKNQFSLQLNSVTAADTAIYYCTSXXXXXXXXXXXWKGDVWGKGTTVTVSSGGGGSEIVMTQSPGTLSLSPGERATLSCXXXXXXXXXXXXXXWFQQKPGQAPRLLIYXXXXXXXXXGFPDRFSGSGSGTDFTLTITRLEPEDFAMYYCXXXXXXXXXXXFGQGTKLEIKDYKDDDDK

SEQ ID NO: 50 - protein, 402 aa, Artificial Sequence
  Description of Artificial Sequence Synthetic polypeptide
  MOD_RES (31)..(37): Any amino acid
  MISC_FEATURE (31)..(37): This region may encompass 3-7 residues
  MOD_RES (52)..(62): Any amino acid
  MISC_FEATURE (52)..(62): This region may encompass 7-11 residues
  MOD_RES (95)..(105): Any amino acid
  MISC_FEATURE (95)..(105): This region may encompass 7-11 residues
  MOD_RES (150)..(163): Any amino acid
  MISC_FEATURE (150)..(163): This region may encompass 9-14 residues
  MOD_RES (179)..(187): Any amino acid
  MISC_FEATURE (179)..(187): This region may encompass 5-9 residues
  MOD_RES (220)..(230): Any amino acid
  MISC_FEATURE (220)..(230): This region may encompass 7-11 residues
  See specification as filed for detailed description of substitutions and preferred embodiments
QVQLQQWGAGLLKPSETLRLTCAVYGGSFTXXXXXXXWIRQPPGKGLEWIGXXXXXXXXXXXRVTISVDTSKNQFSLQLNSVTAADTAIYYCTSXXXXXXXXXXXWKGDVWGKGTTVTVSSGGGGSEIVMTQSPGTLSLSPGERATLSCXXXXXXXXXXXXXXWFQQKPGQAPRLLIYXXXXXXXXXGFPDRFSGSGSGTDFTLTITRLEPEDFAMYYCXXXXXXXXXXXFGQGTKLEIKGGGGSGGGGSGGGGSTDQCDNFPQMLRDLRDAFSRVKTFFQTKDELDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR

SEQ ID NO: 51 - protein, 409 aa, Artificial Sequence
  Description of Artificial Sequence Synthetic polypeptide
  MOD_RES (192)..(198): Any amino acid
  MISC_FEATURE (192)..(198): This region may encompass 3-7 residues
  MOD_RES (213)..(223): Any amino acid
  MISC_FEATURE (213)..(223): This region may encompass 7-11 residues
  MOD_RES (256)..(266): Any amino acid
  MISC_FEATURE (256)..(266): This region may encompass 7-11 residues
  MOD_RES (311)..(324): Any amino acid
  MISC_FEATURE (311)..(324): This region may encompass 9-14 residues
  MOD_RES (340)..(348): Any amino acid
  MISC_FEATURE (340)..(348): This region may encompass 5-9 residues
  MOD_RES (381)..(391): Any amino acid
  MISC_FEATURE (381)..(391): This region may encompass 7-11 residues
  See specification as filed for detailed description of substitutions and preferred embodiments
TDQCDNFPQMLRDLRDAFSRVKTFFQTKDELDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKARGGGGSGGGGSGGGGSQVQLQQWGAGLLKPSETLRLTCAVYGGSFXXXXXXXWIRQPPGKGLEWIGXXXXXXXXXXXRVTISVDTSKNQFSLQLNSVTAADTAIYYCTSXXXXXXXXXXXWKGDVWGKGTTVTVSSGGGGSEIVMTQSPGTLSLSPGERATLSCXXXXXXXXXXXXXXWFQQKPGQAPRLLIYXXXXXXXXXGFPDRFSGSGSGTDFTLTITRLEPEDFAMYYCXXXXXXXXXXXFGQGTKLEIKDYKDDDDK

SEQ ID NO: 52 - DNA, 19 bp, Homo sapiens
ggcgggcagggcctgaagt

SEQ ID NO: 53 - DNA, 13 bp, Homo sapiens
cagagtagctgaa

SEQ ID NO: 54 - DNA, 10 bp, Homo sapiens
tcggcggcgg

SEQ ID NO: 55 - DNA, 10 bp, Homo sapiens
ctcctcccct

SEQ ID NO: 56 - DNA, 13 bp, Homo sapiens
tagcagcagcagc

SEQ ID NO: 57 - DNA, 11 bp, Homo sapiens
ctttttttttt

SEQ ID NO: 58 - DNA, 21 bp, Homo sapiens
cactgactgagatgttctctt

SEQ ID NO: 59 - DNA, 15 bp, Homo sapiens
aaattttgaagtgtt

SEQ ID NO: 60 - DNA, 10 bp, Homo sapiens
cgcagcgcca

SEQ ID NO: 61 - DNA, 10 bp, Homo sapiens
cggcccccca

SEQ ID NO: 62 - DNA, 16 bp, Homo sapiens
ttgtggacagggagtg

SEQ ID NO: 63 - DNA, 10 bp, Homo sapiens
ttgggggagg

SEQ ID NO: 64 - DNA, 19 bp, Homo sapiens
attttcttttctttctttc

SEQ ID NO: 65 - DNA, 12 bp, Homo sapiens
gtcaacatttat

SEQ ID NO: 66 - DNA, 18 bp, Homo sapiens
tacatgagttggtgcagg

SEQ ID NO: 67 - DNA, 11 bp, Homo sapiens
ccagaatggca

SEQ ID NO: 68 - DNA, 16 bp, Homo sapiens
gtaacctttaactcaa

SEQ ID NO: 69 - DNA, 11 bp, Homo sapiens
taaaacaaaac